(19) United States
(12) Patent Application Publication
(10) Pub. No.: US 2026/0069408 A1
(43) Pub. Date: Mar. 12, 2026

(54) DEVICE FOR CAPTURING INNER SURFACES OF LEAFLETS OF A HEART VALVE PROSTHESIS DURING CRIMPING

(71) Applicant: MEDTRONIC, INC., Minneapolis, MN (US)

(72) Inventors: Ciara HAREN, Santa Rosa, CA (US); Brian J. CASTELLI, Santa Rosa, CA (US); Steven CLAESSENS, Santa Rosa, CA (US); Matthew BALDWIN, Santa Rosa, CA (US)

(21) Appl. No.: 18/867,978

(22) PCT Filed: Jun. 5, 2023
(86) PCT No.: PCT/IB2023/055768

§ 371 Date: Nov. 21, 2024

Related U.S. Application Data
(60) Provisional application No. 63/350,587, filed on Jun. 9, 2022.

(51) Int. Cl. A61F 2/24

(52) U.S. Cl. CPC A61F 2/2418

(57) ABSTRACT

An assembly including a transcatheter valve prosthesis and a crimping accessory. The transcatheter valve prosthesis including a stent and a valve component including at least one leaflet secured to the stent. The valve prosthesis includes a crimped having a crimped configuration for delivery and an expanded configuration. The crimping accessory assists in transitioning the valve prosthesis into the crimped configuration. In a first embodiment, the crimping accessory includes a conduit having a vacuum port configured to use suction force to move at least one leaflet radially inward. In a second embodiment, the crimping accessory includes a plurality of rods or graspers configured to contact and hold the at least one leaflet and to place a radially inward force as to move the least one leaflet radially inwards. Once each of the embodiments applies a force, the valve prosthesis is partially radially compressed into the crimped configuration.

### FIELD OF THE INVENTION

The present technology is generally related to heart valve prosthetics and is directed towards protecting the prosthetic leaflets during the crimping process.

### BACKGROUND

The human heart is a four chambered, muscular organ that provides blood circulation through the body during a cardiac cycle. The four main chambers include the right atrium and right ventricle which supplies the pulmonary circulation, and the left atrium and left ventricle which supplies oxygenated blood received from the lungs into systemic circulation. To ensure that blood flows in one direction through the heart, atrioventricular valves (tricuspid and mitral valves) are present between the junctions of the atrium and the ventricles, and semi-lunar valves (pulmonary valve and aortic valve) govern the exits of the ventricles leading to the lungs and the rest of the body. These valves contain leaflets or cusps that open and shut in response to blood pressure changes caused by the contraction and relaxation of the heart chambers. The valve leaflets move apart from each other to open and allow blood to flow downstream of the valve, and coapt to close and prevent backflow or regurgitation in an upstream manner.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

The present disclosure relates to improvements in radially compressing or crimping a heart valve prosthesis to ensure that the heart valve prosthesis has a low profile for transcatheter delivery through a patient's vasculature and further to ensure that the leaflets of the heart valve prosthesis are not damaged during crimping.

### BRIEF SUMMARY OF THE INVENTION

According to a first embodiment hereof, the present disclosure provides an assembly including a transcatheter valve prosthesis and a crimping accessory configured for use when radially compressing the transcatheter valve prosthesis into the crimped configuration. The transcatheter valve prosthesis includes a stent and a prosthetic valve component including at least one leaflet positioned within and secured to the stent. The transcatheter valve prosthesis has a crimped configuration for delivery within a vasculature and an expanded configuration. The crimping accessory includes at least one conduit. Each conduit includes a first end, a second end having at least one vacuum port configured to contact an inner surface of the at least one leaflet, and a lumen extending from the first end to the second end. A manifold is coupled to the at least one conduit. The manifold is configured to be fluidly coupled to a suction source such that the lumen of the at least one conduit and the at least one vacuum port is also fluidly coupled to the suction source. The at least one conduit is configured to move radially, and the crimping accessory is configured to apply a suction force to the inner surface of the at least one leaflet, the suction force applying an inward radial force to move the at least one leaflet radially inwards while the transcatheter valve prosthesis is being radially compressed.

In an aspect of the first embodiment, and in combination with any other aspects herein, the disclosure provides that the at least one conduit includes exactly three conduits.

In an aspect of the first embodiment, and in combination with any other aspects herein, the disclosure provides that the at least one conduit includes a plurality of conduits, and the plurality of conduits are circumferentially spaced apart from each other at substantially equal intervals.

In an aspect of the first embodiment, and in combination with any other aspects herein, the disclosure provides that the manifold is configured to be disposed over a delivery system.

In an aspect of the first embodiment, and in combination with any other aspects herein, the disclosure provides that the crimping accessory further includes a compression ring which is slidably disposed over an outer surface of the manifold and is configured to move axially relative to the manifold.

In an aspect of the first embodiment, and in combination with any other aspects herein, the disclosure provides that movement of the compression ring relative to the manifold controls radial movement of the at least one conduit.

In an aspect of the first embodiment, and in combination with any other aspects herein, the disclosure provides that the second end of the at least one conduit is curved.

In an aspect of the first embodiment, and in combination with any other aspects herein, the disclosure provides that the second end of the at least one conduit has a larger diameter than a diameter of a body of the at least one conduit.

In an aspect of the first embodiment, and in combination with any other aspects herein, the disclosure provides that the second end of the at least one conduit is paddle shaped.

In an aspect of the first embodiment, and in combination with any other aspects herein, the disclosure provides that the at least one conduit is curved.

In an aspect of the first embodiment, and in combination with any other aspects herein, the disclosure provides that the crimping accessory further includes a collar positioned in axial alignment with the manifold.

In an aspect of the first embodiment, and in combination with any other aspects herein, the disclosure provides that the collar is slidable relative to the at least one conduit and axial movement of the collar controls radial movement of the at least one conduit.

In an aspect of the first embodiment, and in combination with any other aspects herein, the disclosure provides that the collar includes at least one channel configured to house a portion of at least one conduit.

In an aspect of the first embodiment, and in combination with any other aspects herein, the disclosure provides that the crimping accessory further includes a first configuration wherein the second end of the at least one conduit is positioned within at least one channel of the collar.

In an aspect of the first embodiment, and in combination with any other aspects herein, the disclosure provides that the crimping accessory further includes a second configuration wherein the second end of the at least one conduit is positioned radially external relative to the second body.

In an aspect of the first embodiment, and in combination with any other aspects herein, the disclosure provides that the at least one conduit extends perpendicularly from a surface of the collar when the crimping accessory is in the second configuration.

According to a second embodiment hereof, the present disclosure provides a method for crimping a transcatheter valve prosthesis. A crimping accessory is positioned adjacent to a transcatheter valve prosthesis. The transcatheter valve prosthesis is disposed within a crimper chamber of a crimper. The crimping accessory includes at least one conduit having a first end, a second end, and a lumen extending from the first end to the second end, the second end having at least one vacuum port. The second end of the at least one conduit is positioned in contact with an inner surface of at least one leaflet of the transcatheter valve prosthesis. The crimping accessory is coupled to a suction source. A suction force from the suction source is applied to the inner surface of the at least one leaflet as to move the at least one leaflet radially inwards. The transcatheter valve prosthesis is partially crimped while maintaining the suction force.

In an aspect of the second embodiment, and in combination with any other aspects herein, the disclosure provides that application of the suction force is ceased and the crimping accessory is removed from being in contact with the at least one leaflet of the transcatheter valve prosthesis. The transcatheter valve prosthesis is further compressed to the crimped configuration.

According to a third embodiment hereof, the present disclosure provides an assembly including a transcatheter valve prosthesis and a crimping accessory configured for use when radially compressing the transcatheter valve prosthesis into the crimped configuration. The transcatheter valve prosthesis includes a stent and a prosthetic valve component including at least one leaflet positioned within and secured to the stent. The transcatheter valve prosthesis has a crimped configuration wherein the transcatheter valve prosthesis is crimped to the catheter delivery system and an expanded configuration for deployment within a native heart valve. The crimping accessory includes a plurality of rods configured to contact and hold the at least one leaflet and to place a radially inward force as to move the least one leaflet radially inwards and a hub operably coupled to an end of each of the plurality of rods. Each rod of the plurality of rods is configured to move radially relative to the hub.

In an aspect of the third embodiment, and in combination with any other aspects herein, the disclosure provides that the plurality of rods includes exactly nine rods.

In an aspect of the third embodiment, and in combination with any other aspects herein, the disclosure provides that the hub of the crimping accessory is configured to be coupled to the delivery system while compressing the transcatheter valve prosthesis into the crimped state.

In an aspect of the third embodiment, and in combination with any other aspects herein, the disclosure provides that the leaflet folding assembly further includes a compression ring which is slidably disposed over an outer surface of the hub and is configured to move axially relative to the hub.

In an aspect of the third embodiment, and in combination with any other aspects herein, the disclosure provides that movement of the compression ring relative to the hub controls radial movement of the plurality of rods.

In an aspect of the third embodiment, and in combination with any other aspects herein, the disclosure provides that exactly three rods are configured to hold a single leaflet, the exactly three rods including a first rod, a second rod and a third rod, the second rod being disposed between the first and third rods.

In an aspect of the third embodiment, and in combination with any other aspects herein, the disclosure provides that the first and third rods are configured to contact an inner surface of the single leaflet and the second rod is configured to contact an outer surface of the single leaflet.

In an aspect of the third embodiment, and in combination with any other aspects herein, the disclosure provides that the second rod is removably coupled to the hub.

In an aspect of the third embodiment, and in combination with any other aspects herein, the disclosure provides that the first and second rods are configured to contact an inner surface of the single leaflet and the third rod is configured to contact an outer surface of the single leaflet.

In an aspect of the third embodiment, and in combination with any other aspects herein, the disclosure provides that the compression sleeve includes an angled track on an inner surface thereof for receiving the third rod, the angled track being configured to guide and deflect the third rod laterally towards the first and second rods during relative axial movement between the compression sleeve and the hub.

According to a fourth embodiment hereof, the present disclosure provides a method for crimping a transcatheter valve prosthesis onto a delivery system. A crimping accessory is positioned to be in contact with at least one leaflet of a transcatheter valve prosthesis, the transcatheter valve prosthesis being disposed within a crimper chamber. The crimping accessory has a plurality of rods. The at least one leaflet is gripped with the plurality of rods. An inward radial force is applied with the plurality of rods to the at least one leaflet as to move the at least one leaflet radially inwards. The transcatheter valve prosthesis is partially crimped while maintaining the radially inward force to the at least one leaflet.

In an aspect of the fourth embodiment, and in combination with any other aspects herein, the disclosure provides that the crimping accessory is removed from being in contact with the at least one leaflet of the transcatheter valve prosthesis. The transcatheter valve prosthesis is compressed from a partially crimped configuration to a fully crimped configuration.

According to a fifth embodiment hereof, the present disclosure provides an assembly including a transcatheter valve prosthesis and a crimping accessory configured for use when radially compressing the transcatheter valve prosthesis into the crimped configuration. The transcatheter valve prosthesis includes a stent and a prosthetic valve component including at least one leaflet positioned within and secured to the stent. The transcatheter valve prosthesis has a crimped configuration for delivery within a vasculature and an expanded configuration. The crimping accessory includes a body and a plurality of graspers positioned at the body. Each grasper is configured to contact exactly one leaflet. Each grasper is positioned at an outer surface of the at least one leaflet. The crimping accessory is configured to contact and hold the at least one leaflet radially inward while the transcatheter valve prosthesis is compressed into the crimped configuration.

In an aspect of the fifth embodiment, and in combination with any other aspects herein, the disclosure provides that the body is annular.

In an aspect of the fifth embodiment, and in combination with any other aspects herein, the disclosure provides that the plurality of graspers are circumferentially spaced apart from each other at substantially equal intervals.

In an aspect of the fifth embodiment, and in combination with any other aspects herein, the disclosure provides that the each grasper of the plurality of graspers further includes a recess, the recess being configured to accept a finger of a user.

In an aspect of the fifth embodiment, and in combination with any other aspects herein, the disclosure provides that each grasper of the plurality of graspers is operatively coupled to the body as to freely rotate relative to the body.

In an aspect of the fifth embodiment, and in combination with any other aspects herein, the disclosure provides that each grasper of the plurality of graspers may rotate independently from one another.

According to a sixth embodiment hereof, the present disclosure provides an assembly including a method for crimping a transcatheter valve prosthesis onto a delivery system. A crimping accessory is positioned adjacent to a transcatheter valve prosthesis. The transcatheter valve prosthesis is disposed within a crimper chamber of a crimper. The crimping accessory includes a body and a plurality of graspers positioned at the body. The plurality of graspers is positioned as to be in contact with at least one leaflet of the transcatheter valve prosthesis. The plurality of graspers are rotated relative to the body as to move the at least one leaflet radially inwards. The transcatheter valve prosthesis is partially compressed while maintaining the radial position of the at least one leaflet.

In an aspect of the sixth embodiment, and in combination with any other aspects herein, the disclosure provides that the crimping accessory is removed from being in contact with the at least one leaflet of the transcatheter valve prosthesis. The transcatheter valve prosthesis is compressed to the crimped configuration.

### BRIEF DESCRIPTION OF DRAWINGS

The foregoing and other features and advantages of the invention will be apparent from the following description of embodiments thereof as illustrated in the accompanying drawings. The accompanying drawings, which are incorporated herein and form a part of the specification, further serve to explain the principles of the invention and to enable a person skilled in the art to make and use the invention. The drawings are not to scale.

FIG. 1A depicts a side view of a transcatheter valve prosthesis in accordance with an aspect of the disclosure.

FIG. 1B depicts a top view of the transcatheter valve prosthesis of FIG. 1A in accordance with an aspect of the disclosure.

FIG. 2 depicts a perspective view of an assembly including a delivery system, the transcatheter valve prosthesis of FIG. 1, and a crimping accessory in accordance with an aspect of the disclosure, wherein the crimping accessory is in an open configuration.

FIG. 3 depicts another perspective view of the assembly of FIG. 2, wherein the crimping accessory is in a closed configuration.

FIG. 4A depicts a sectional view of the crimping accessory of FIG. 2, wherein the crimping accessory is in the open configuration.

FIG. 4B depicts a sectional view of the crimping accessory of FIG. 2, wherein the crimping accessory is the closed configuration.

FIG. 5A depicts a perspective view of a second end of a conduit of the crimping accessory of FIG. 2.

FIG. 5B depicts a perspective view of a second end of a conduit of a crimping accessory according to another embodiment hereof.

FIG. 5C depicts a perspective view of a second end of a conduit of a crimping accessory according to another embodiment hereof.

FIG. 6 depicts a schematic side view of a vacuum source, the transcatheter valve prosthesis of FIG. 1, the crimping accessory of FIG. 2, and the delivery system of FIG. 2.

FIG. 7 depicts a perspective view of a second embodiment of a crimping accessory in accordance with an aspect of the disclosure, wherein the crimping accessory is in a closed configuration.

FIG. 8 depicts a perspective view of the second embodiment of the crimping accessory of FIG. 7 in an open configuration.

FIG. 9 depicts an internal perspective view of the second embodiment of the crimping accessory of FIG. 7 in the closed configuration.

FIG. 10 depicts an internal perspective view of the second embodiment of the crimping accessory of FIG. 7 in the open configuration.

FIG. 11 depicts a perspective view of a conduit of FIG. 7 in the open configuration.

FIG. 12 depicts a sectional view of the crimping accessory of FIG. 7 in the open configuration.

FIG. 13 depicts a perspective view of a third embodiment of a crimping accessory in accordance with an aspect of the disclosure, wherein the crimping accessory is in an open configuration.

FIG. 14 depicts a side view of the third embodiment of the crimping accessory of FIG. 13 in the open configuration.

FIG. 15 depicts a side view of the third embodiment of the crimping accessory of FIG. 13 in a closed configuration.

FIG. 16A depicts a fourth embodiment of a crimping accessory.

FIG. 16B depicts a close-up side view of the crimping accessory of FIG. 16A interacting with a prosthetic valve component.

FIG. 16C depicts another close-up side view of the crimping accessory of FIG. 16A interacting with the prosthetic valve component.

FIG. 16D depicts a close-up top view of the crimping accessory of FIG. 16A interacting with the prosthetic valve component.

FIG. 17A depicts a fifth embodiment of a crimping accessory.

FIG. 17B depicts a close-up top view of the crimping accessory of FIG. 17A interacting with a prosthetic valve component.

FIG. 17C depicts a close-up top view of the crimping accessory of FIG. 17A interacting with the prosthetic valve component.

FIG. 17D depicts a close-up top view of the crimping accessory of FIG. 17A interacting with the prosthetic valve component.

FIG. 18 depicts a perspective view of a sixth embodiment of a crimping accessory in accordance with an aspect of the disclosure, wherein the crimping accessory is in an open configuration.

FIG. 19 depicts a perspective view of the crimping accessory of FIG. 18 in a closed configuration.

FIG. 20 depicts a perspective view of a grasper of the crimping accessory of FIG. 18.

FIG. 21 depicts a method for using a crimping accessory and a crimper to crimp a transcatheter valve prosthesis.

### DETAILED DESCRIPTION OF THE INVENTION

Specific embodiments of the present invention are now described with reference to the figures, wherein like reference numbers indicate identical or functionally similar elements. The terms “inflow” and “outflow”, when used in the following description to refer to a native vessel, native valve, or a device to be implanted into a native vessel or native valve, such as a transcatheter valve prosthesis, are with reference to the direction of blood flow. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention of the application and uses of the invention. Furthermore, there is no intention to be bound by any expressed or implied theory presented in the preceding field of the invention, background, summary or the following detailed description.

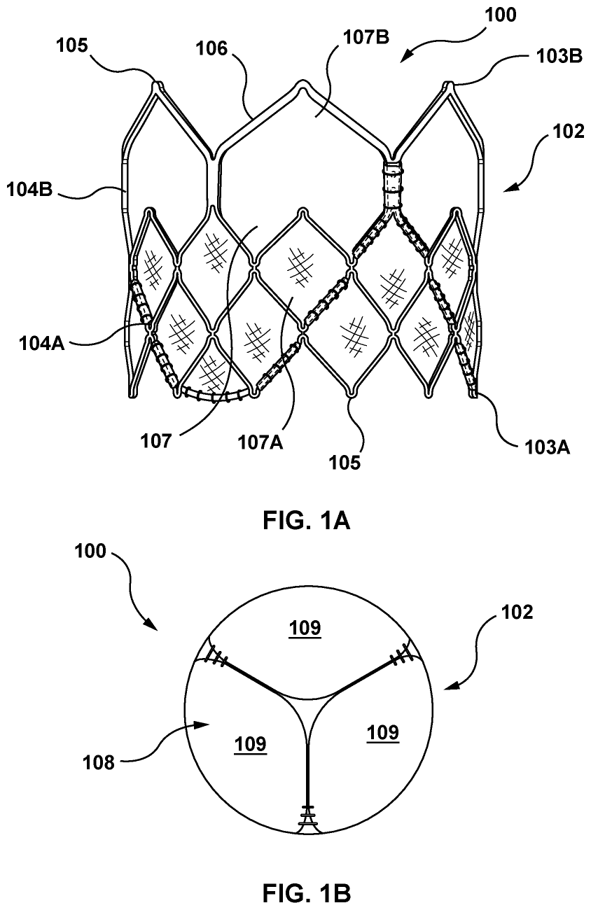

FIGS. 1A and 1B illustrate a transcatheter valve prosthesis 100 that may be utilized with the embodiments of the crimping accessory described herein. The transcatheter valve prosthesis 100 is illustrated herein in order to facilitate description of the present invention. The following description of the transcatheter valve prosthesis 100 is merely exemplary in nature and is not intended to limit the invention or the application and uses of the invention. It is understood that any number of alternate heart valve prostheses can be used with the leaflet accessory tools and methods described herein. Other non-limiting examples of transcatheter valve prostheses that can be used with the leaflet accessory tools described herein are described in U.S. Patent App. Ser. No. 17/186,485, filed Feb. 26, 2021, which is incorporated by reference herein in its entirety. Although the transcatheter valve prosthesis 100 is a balloon-expandable heart valve prosthesis configured for placement within an aortic heart valve, embodiments of the crimping accessory described herein may be utilized with any transcatheter valve prosthesis that is crimped onto a delivery system. For example, embodiments of the crimping accessory described herein may be utilized with a transcatheter heart valve configured for placement within a pulmonary, aortic, mitral, or tricuspid valve, or may be utilized with a transcatheter valve prosthesis configured for placement within a venous valve or within other body passageways where it is deemed useful. Embodiments of the crimping accessory described herein may be utilized with a self-expanding transcatheter valve prosthesis or a balloon-expandable transcatheter valve prosthesis. There is no intention to be bound by any expressed or implied theory presented in the preceding technical field, background, brief summary or the following detailed description.

The transcatheter valve prosthesis 100 includes a radially-expandable frame or stent 102 and a prosthetic valve component 108. The stent 102 is generally tubular and is balloon expandable, having a crimped configuration for delivery within a vasculature and an expanded configuration for deployment within a native heart valve. FIG. 1A is a side view of the transcatheter valve prosthesis 100 in the expanded configuration, while FIG. 1B is a top view of the transcatheter valve prosthesis 100. In an embodiment, the transcatheter valve prosthesis 100 is configured for replacement of an aortic valve such that an inflow end 103A of the transcatheter valve prosthesis 100 extends into and anchors within the aortic annulus of a patient's left ventricle, while an outflow end 103B of the transcatheter valve prosthesis 100 is positioned within the aortic sinuses.

The stent 102 of the transcatheter valve prosthesis 100 may be a unitary frame or scaffold that supports the prosthetic valve component 108 disposed or positioned within the interior of the stent 102. The prosthetic valve component 108 of the transcatheter valve prosthesis 100 is capable of regulating flow therethrough via valve leaflets 109 that may form a replacement valve. FIGS. 1A and 1B illustrate an exemplary valve component 108 having three leaflets 109, although a single leaflet or bicuspid leaflet configuration may alternatively be used in embodiments hereof. The valve leaflets 109 may be attached to the stent 102 using sutures or otherwise and sealingly attached along their bases to portions of the stent 102. Adjoining pairs of leaflets 109 are attached to one another at their lateral ends to form commissures, with free edges that meet in an area of coaptation. The orientation of the leaflets 109 within the valve component 108 depends upon on which end of the transcatheter valve prosthesis 100 is the inflow end and which end of the transcatheter valve prosthesis 100 is the outflow end, thereby ensuring one-way flow of blood through the transcatheter valve prosthesis 100.

The valve leaflets 109 may be formed of various flexible materials including, but not limited to natural pericardial material such as tissue from bovine, equine or porcine origins, or synthetic materials such as polytetrafluoroethylene (PTFE), DACRON® polyester, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject to stretching, deforming, or fatigue.

The stent 102, and the transcatheter valve prosthesis 100, has a crimped configuration for delivery within a vasculature and an expanded configuration (as shown in FIGS. 1A and 1B) for deployment within a native heart valve. The crimped configuration as used herein refers to the configuration of the stent 102 or the transcatheter valve prosthesis 100 after crimping onto a balloon of a delivery system. The expanded configuration as used herein refers to the configuration of the stent 102 or the transcatheter valve prosthesis 100 after radial expansion by the balloon.

The stent 102 includes an inflow portion 104A and an outflow portion 104B. The inflow portion 104A is formed proximate to the inflow end 103A of the stent 102. The inflow portion 104A includes a plurality of crowns 105 and a plurality of struts 106 with each crown 105 being formed between a pair of opposing struts 106. Each crown 105 is a curved segment or bend extending between opposing struts 106. The inflow portion 104A is generally tubular, with a plurality of side openings being defined by the plurality of crowns 105 and the plurality of struts 106. Referring to FIG. 1A, the side openings include a plurality of inflow side openings 107A and a plurality of outflow side openings 107B, which are collectively referred to herein as side openings 107. The inflow side openings 107A are diamond shaped and are covered by a skirt or graft material, while the outflow side openings 107B may be open or, in other words, uncovered by such a skirt or graft material. The outflow side openings 107B are relatively larger than the plurality of side openings 107A to improve access to the coronary arteries. More particularly, the outflow side openings 107B are configured to be of sufficient size to be easily crossed with a coronary guide catheter into either the right coronary artery or the left main coronary artery once the transcatheter heart valve 100 is deployed in situ.

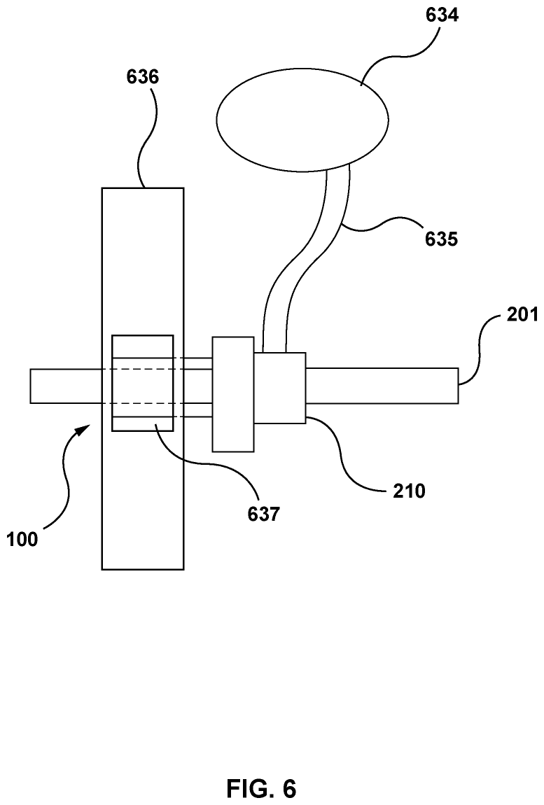

A crimper may be used to radially compress the transcatheter valve prosthesis 100 from the expanded configuration to the crimped configuration on a balloon of a delivery system for delivery within a vasculature. Crimping the transcatheter valve prosthesis 100 is accomplished by placing the transcatheter valve prosthesis 100, when in the expanded configuration, onto the desired location on the delivery system 201, and placing the delivery system 201 and the transcatheter valve prosthesis 100 into a crimper, as further described herein with respect to FIG. 6. During the crimping process, the prosthetic valve component 108 may protrude or extend through the side openings 107 of the stent 102, particularly the outflow side openings 107B, and, as a result, become pinched therein. This may lead to damage of the leaflets 109, such as splitting, peeling, experiencing partial or full cuts, or separation of the layers of material. Each of these types of damages is considered unacceptable and may render the entire transcatheter valve prosthesis defective. Further, even if the leaflets 109 are not damaged as described above, they may still extend through the side openings 107 of the stent 102 resulting in the leaflets 109 protruding radially outside of the stent 109. If protruded, the leaflets 109 may extend radially far enough to exceed the profile tolerance of the transcatheter valve prosthesis 100, which may undesirably increase a crossing profile of the transcatheter valve prosthesis 100. Therefore, there exists a need to ensure the valve component 108 is maintained within the stent 102 during the crimping process. Embodiments hereof relate to crimping accessories for use when radially compressing the transcatheter valve prosthesis 100 into the crimped configuration. In particular, the crimping accessories described herein hold and ensure that the prosthetic valve component 108 is maintained within the stent 102 as to prevent or limit any potential damage or protrusion of the prosthetic valve component 108.

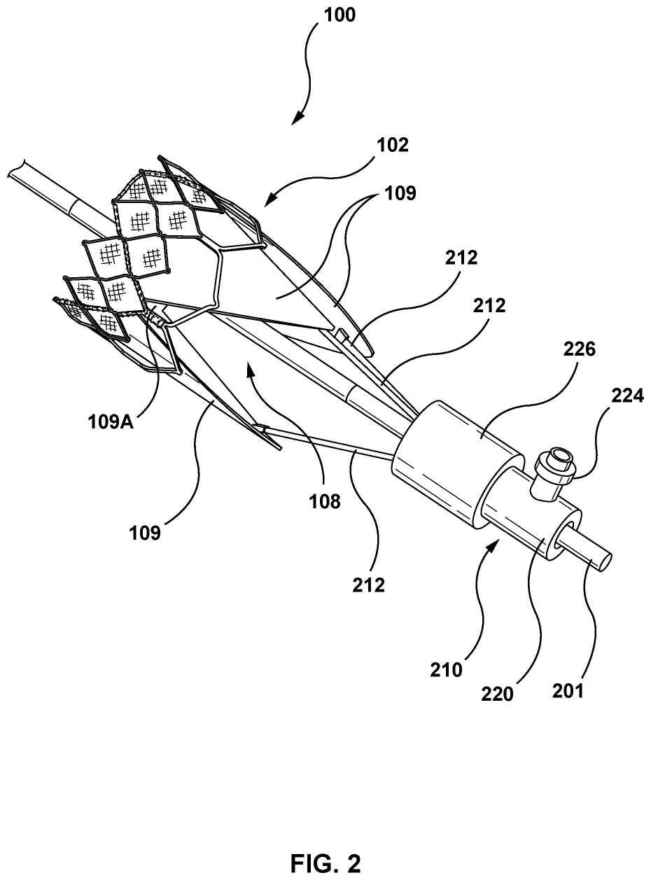

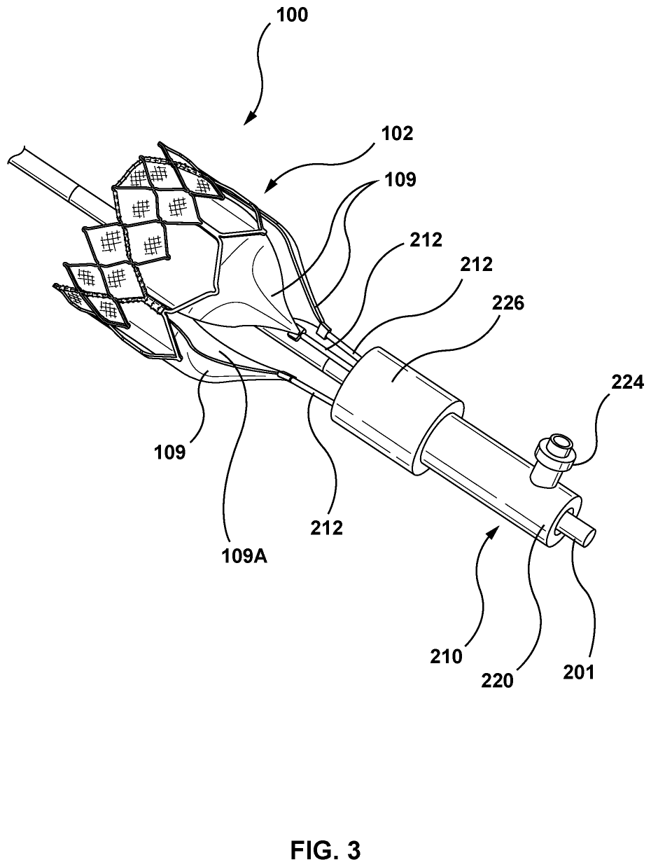

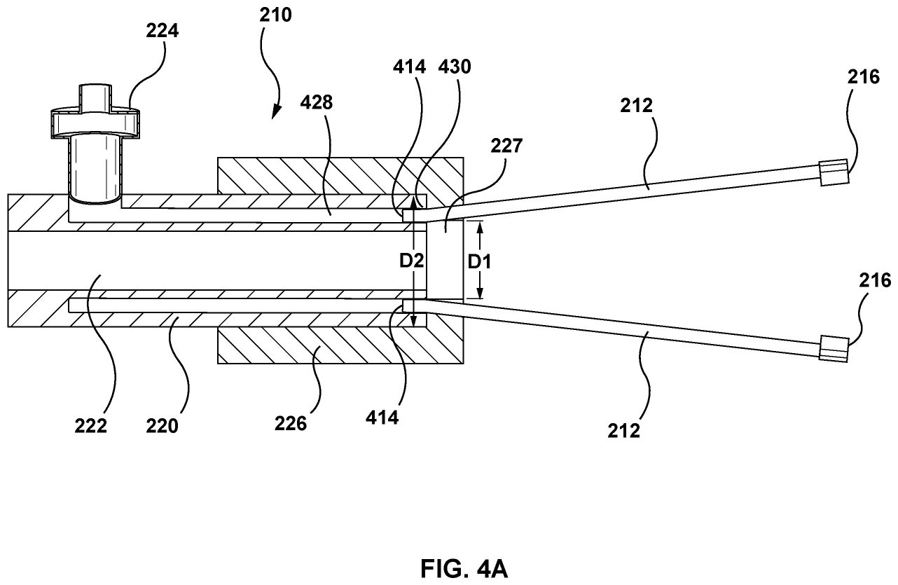

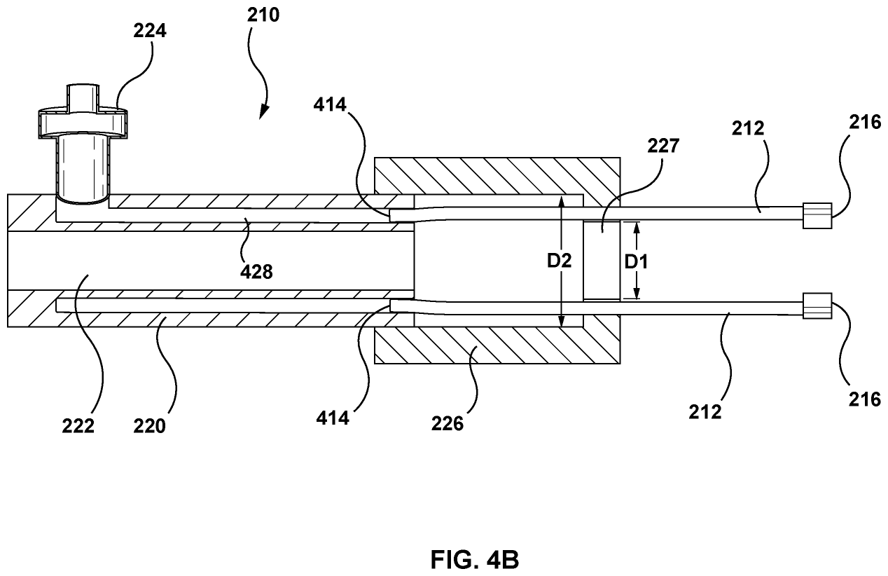

With reference to FIGS. 2-4, a first embodiment of a crimping accessory 210 is described. The crimping accessory 210 is a tool that is configured for use with a transcatheter valve prosthesis (such as but not limited to the transcatheter valve prosthesis 100) when radially compressing the transcatheter valve prosthesis into a crimped configuration for delivery within a vasculature. The crimping accessory 210 includes an open configuration, which is shown in FIG. 2 and FIG. 4A, and a closed configuration, which is shown in FIG. 3 and FIG. 4B. Additionally, FIG. 2 and FIG. 3 show the crimping accessory 210 interacting with the transcatheter valve prosthesis 100, while FIG. 4A and FIG. 4B show a side sectional views of the crimping accessory 210. For illustrative purposes only, the crimping accessory 210 will be described for use with the transcatheter valve prosthesis 100 since the structure thereof has been previously described herein.

The crimping accessory 210 includes a manifold 220, a plurality of conduits 212 coupled to and extending distally from the manifold 220, and a compression ring 226 disposed over the manifold 220. Generally, the manifold 220 is configured to provide a suction or vacuum force through the conduits 212 and to the leaflets 109. More particularly, the manifold 220 includes a cylindrical body which has a lumen 222 extending therethrough. The lumen 222 of the manifold 220 is configured as to allow for the manifold 220 to be slidingly positioned over a delivery system 201, as shown in FIG. 2 and FIG. 3. The manifold 220 also includes a coupler 224 which is configured to fluidly connect the crimping accessory 210 to a vacuum or suction source (not shown in FIG. 2). Stated another way, the vacuum or suction source may be connected or removably attached to the coupler 224. As best shown on FIGS. 4A and 4B, the manifold 220 additionally includes a plurality of channels 428 that extend through the manifold 220 as to fluidly couple the coupler 224 to each conduit 212. Below the coupler 224, an annular channel is formed within the sidewall of the manifold 220 such that the plurality of channels 428 merge into the annular channel and therefore are in fluid communication with each other as well as in fluid communication with the coupler 224. Additionally, the manifold 220 includes a plurality of conduit hinges 430, with each conduit hinge 430 functioning to attach or secure a conduit 212 to the manifold 220 while permitting the conduits 212 to be deflected or moved radially inwards as described in more detail herein.

Each conduit 212 has a first end 414 which is disposed within and coupled to the manifold 220 via a conduit hinge 430 and a second end 216 which includes a vacuum port 218 configured to contact a leaflet 109 of the transcatheter valve prosthesis 100. Additionally, each conduit 212 includes a lumen which extends from the first end 414 to the second end 216 allowing for each conduit 212 to apply the suction force to the leaflets 109. More particularly, each conduit 212 is configured to contact an inner surface 109A of the leaflets 109 to apply the suction force as to move or displace the leaflets 109 radially inward and away from the side openings 107 of the stent 102. The conduits 212 are configured to contact only the inner surfaces of the leaflets 109 of the valve component 108. Stated another way, each conduit 212 is radially disposed within the leaflets 109 of the valve component 108 and is configured such that the conduit 212 does not directly contact the outer surface of the leaflets 109 and does not directly contact the stent 102 of the transcatheter valve prosthesis 100.

Each conduit 212 is formed of a flexible material. The material of the plurality of conduits 212 must have sufficient stiffness to extend into the transcatheter valve prosthesis 100 and thereby contact the leaflets 109 when the transcatheter valve prosthesis 100 is compressed into the crimped configuration, but must also be configured to contact the leaflets 109 without causing damage thereto. Suitable materials for the plurality of conduits 212 include but are not limited to polyimide, Nitinol, or thin-walled stainless steel.

As best shown on FIGS. 2 and 3, the plurality of conduits 212 are circumferentially spaced apart from each other. In an embodiment, the plurality of conduits 212 include at least three conduits. As shown in the embodiment of FIGS. 2-6, the plurality of conduits 212 may include exactly three conduits 212, with each conduit being configured to contact a single leaflet 109 of the valve component 108. However, in another embodiment, the plurality of conduits 212 may include more than three conduits. The number of conduits 212 may depend on the number of leaflets 109 and/or the size and configuration of the stent 102. For example, the plurality of conduits 212 may include six conduits 212, with two conduits 212 being configured to contact a single leaflet 109 of the valve component 108. When configured for use with a valve component having single leaflet or bicuspid leaflet configuration, the number of conduits 212 configured to contact each leaflet may vary from the tricuspid leaflet configuration. When the crimping tool 210 includes exactly three conduits 212, and the transcatheter valve prosthesis 100 includes exactly three leaflets, the conduits 212 are circumferentially spaced apart from each other at substantially equal intervals with a single conduit being positioned to contact a single leaflet.

As stated above, the second end 216 of each conduit 212 is configured to contact and hold, using vacuum or suction force, the inner surface 109A of a leaflet 109 and apply the force as to move or displace the leaflet 109 radially inward and away from the side openings 107 of the stent 102. The vacuum port 218 is positioned at the second end 216 of each conduit 212, allowing for the crimping accessory 210 to place the suction force onto the inner surface 109A of the leaflet 109. Each vacuum port 218 is configured to apply a suction force suitable to secure the leaflet 109 to the vacuum port 218. The suitable suction force depends on various factors, such as the material of the leaflet 109 and the size of the vacuum port 218. In an embodiment in which the leaflet material is porcine or bovine material and the vacuum port has an opening of between 0.08 inches and 0.15 inches, a suction force of approximately 5 inHg is sufficient to adhere the leaflet 109 to the vacuum port 218. A singular or a plurality of vacuum ports 218 may be positioned at each of the second ends 216 as to alter the amount of force and surface area of the leaflet 109 that the suction force is applied to. Further, the dimensions of the vacuum port 218 may be altered as to increase or decrease the amount of force placed on the leaflet 109 or to increase or decrease the amount of surface area of the leaflet 109 that is being interacted with.

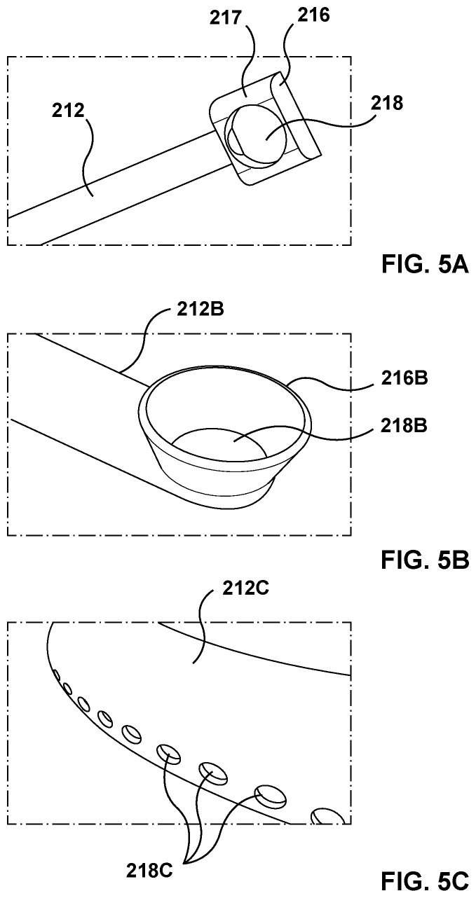

The second end 216 of a conduit 212 is depicted in greater detail in FIG. 5A. In the embodiment of FIG. 5A, the second end 216 has a paddle-shaped or flattened configuration, and is relatively larger in width than a remining length of the conduit 212. The second end 216 includes the vacuum port 218 which is oriented towards, or facing, the inner surface 109A of each leaflet 109. The configuration of the second end 216 is merely exemplary and a variety of alternative configurations may be used to contact the inner surface 109A of the leaflet 109, as shown in FIG. 5B and FIG. 5C. In FIG. 5B, a conduit 212B is shown having a second end 216B with a flared, conical configuration. The second end 216B defines a vacuum port 218B. In FIG. 5C, a conduit 212C is shown having a curved second end 216C with a plurality of vacuum portions 218C defined in or through a sidewall of the conduit 212C. Including multiple vacuum ports 218C increase the amount of contact area between the conduit 212C and the leaflet 109. Further, the second end 216C includes a curved outer surface to provide an atraumatic surface geometry for contacting the inside surface 109A of a leaflet 109.

The crimping accessory 210 is configured to have an open configuration and a closed configuration. While in the open configuration, as shown in FIG. 2 and FIG. 4A, the crimping accessory 210 is configured to initially grab or contact the leaflets 109 of the prosthetic valve component 108. While in the closed configuration, as shown in FIG. 3 and FIG. 4B, the crimping accessory 210 is configured to displace and hold the leaflets 109 radially inward relative to the stent 102. After making contact with the leaflets 109, the crimping accessory 210 may be transitioned from the open configuration to the closed configuration prior to initiation of the crimping process as to move the leaflets 109 away from the stent 102 and, more particularly, the side openings 107 of the stent 102. Alternatively, the crimping accessory 210 may be transitioned from the open configuration to the closed configuration in a gradual manner, during the crimping process. In other words, the crimping accessory 210 may begin in the open configuration at the start of the crimping process and, as the process continues, be transitioned to the closed configuration at a rate that matches the rate of the crimping process.

A compression ring 226 is slidingly disposed over the manifold 220 and is configured to transition the crimping accessory 210 between the open and closed configurations. The compression ring 226 is a tubular component that includes a lumen 227 which extends through the entirety of the compression ring 226. The lumen 227 includes a first diameter D1 at a distal end portion thereof and a second diameter D2 for the remaining length thereof. The first diameter D1 is smaller than the second diameter D2 and is sized as to allow for the delivery device 201 to extend through the length of the compression ring 226. The first diameter D1 has a similar diameter to the lumen 222 of the manifold 220. The second diameter D2 is sized as to allow for the compression ring 226 to slide over an outer surface of the manifold 220. Axial movement or translation of the compression ring 226 is limited due to the stepped transition of the lumen 227 between the first diameter D1 and the second diameter D2, which ensures that the compression ring 226 does not move too far axially towards the manifold 220. Stated another way, when the compression ring 226 is retracted proximally over the manifold 220, the distal end of the manifold 220 abuts against the stepped transition of the lumen 227 between the first diameter D1 and the second diameter D2 and thereby prevents any further proximal retraction of the compression ring 226.

The axial position of the compression ring 226 relative to the manifold 220 controls whether the crimping accessory 210 is in the open configuration or the closed configuration. As shown in FIGS. 4A and 4B, the open configuration occurs when the compression ring 226 is primarily disposed over the manifold 220 rather than the conduits 212, while the closed position occurs when the compression ring 226 is primarily disposed over the conduits rather than the manifold 220. Each of the conduits 212 is preformed or biased to the open configuration, and the compression ring 226 acts to deflect or displace each of the conduits 212 radially inward when the compression ring 226 is advanced thereover. For example, the conduits 212 may be formed in the open configuration from a material such as Nitinol which is biased to return to the open configuration once released from the compression ring 226, or may be formed in the open configuration from a material such as nylon and configured for one-time use. The compression ring 226 thus provides a user or operator with manual control over the radial position of the conduits 212, and thereby provides the operator with manual control over the radial position of the leaflets 109. For example, it may be desirable, due to unforeseen warping or folding of the leaflets 109, that the operator be able to move the radially inward or outward during the crimping process to counteract undesirable outcomes.

Although the conduits 212 are described herein as being biased to the open configuration, other embodiments are contemplated to be within the scope of the present invention. For example, in another embodiment, the conduits 212 may alternatively be preformed or biased to the closed configuration. When formed in the closed configuration, an interior cone-shaped component (not shown) may be slidably disposed within the crimping accessory 210 and may be advanced to deflect or displace each of the conduits 212 radially outward when the interior cone-shaped component is advanced into the conduits 212. In such an embodiment, the conduits 212 may be formed from Nitinol and be biased to return to the closed configuration once the interior cone-shaped component is removed.

To further explain the use of the crimping accessory 210, FIG. 6 depicts a schematic illustration of an assembly including a crimper 636, the crimping accessory 210, the transcatheter valve prosthesis 100, and the delivery system 201. The transcatheter valve prosthesis 100 is disposed or positioned within a crimper chamber 637 of the crimper 636 with the conduits 212 of the crimping accessory 210 positioned radially within the transcatheter valve prosthesis 100 via the outflow portion 104B thereof. Additionally, a vacuum or suction source 634 is positioned externally relative to the crimper 636, crimping accessory 210, and the delivery system 201. The vacuum source 634 is coupled to the coupler 224 of the manifold 220 via vacuum tubing 635. It is envisioned that the delivery system 201 is positioned within the lumen 222 of the manifold 220, and the crimping accessory 210 may be advanced over and slide relative to an outer surface of the delivery system 201.

With the structure of the crimping accessory 210 described in detail above, methods of using the crimping accessory 210 will now be described with reference to FIG. 6. As shown, the crimping accessory 210 is disposed radially within the transcatheter valve prosthesis via the outflow portion 104B thereof. An exemplary delivery system including an expandable balloon for radially expanding the transcatheter valve prosthesis 100 is described in U.S. Application No. Ser. No. 16/908,466, filed Jun. 22, 2021, or U.S. Application Ser. No. 17/406,618, filed Aug. 19, 2021, each of which is herein incorporated by reference in its entirety. However, it will be apparent to one of ordinary skill in the art that other delivery systems may be utilized and that the components of the delivery system may vary depending upon the configuration and structure of the transcatheter valve prosthesis that is being delivered. The crimper 636 may be any mechanical crimping device known in the art that is configured to radially compress the transcatheter valve prosthesis 100 from its expanded configuration to a radially compressed configuration suitable for intravascular delivery as known in the art. For example, the crimper 636 may be, for example, a crimper described in U.S. Patent Appl. No. Ser. No. 17/394,025, filed Aug. 4, 2021, herein incorporated by reference in its entirety.

As shown in FIG. 6, the crimping accessory 210 is configured to be disposed proximally relative to the crimper 636. The transcatheter valve prosthesis 100 is disposed or positioned within the crimper chamber 637 of the crimper 636 with the inflow portion 104A being disposed closer to the distal end of the delivery system 201 than the outflow portion 104B thereof. As such, during crimping of the transcatheter valve prosthesis 100, the conduits 212 extend radially into the stent 102 from the outflow portion 104B thereof. However, it will be understood by one of ordinary skill in the art that the crimping accessory 210 is not required to be slidingly disposed over the delivery system 201 and alternatively may be configured to be disposed over the distal end of the delivery system 201. In such an embodiment, during crimping of the transcatheter valve prosthesis 100, the crimping accessory 210 is configured to be disposed distally relative to the crimper 636 and the conduits 212 extend through the stent 102 of the transcatheter valve prosthesis 100 from the inflow portion 104A thereof.

To position the crimping accessory 210 relative to the transcatheter valve prosthesis 100, the conduits 212 need to be displaced radially inwards prior to being inserted into the stent 102. To do so, the compression ring 226 is advanced distally towards the second ends 216 of the conduits 212, deflecting or moving the conduits 212 radially inwards. When deflected radially inwards, the conduits 212 may be easily positioned into the stent 102 of the transcatheter valve prosthesis 100. Once positioned relative to the stent 102 as desired, a user may proximally retract the compression ring 226 away from the second ends 216 of the conduits 212 as to cause the compression ring 226 to move back to its initial longitudinal position and permit the conduits 212 to resume or revert to their initial radial positions in the open configuration. The vacuum or suction force may then be applied to each of the leaflets 109 via the conduits 212 to couple the conduits 212 to the inner surfaces 109A of the leaflets 109.

After establishing sufficient contact between the conduits 212 and the leaflets 109 via the applied suction force, the crimping accessory 210 may be transitioned from the open configuration to the closed configuration by moving the compression ring 226 distally towards the second ends 216 of the conduits 212, deflecting the conduits 212, and subsequently the leaflets 109, radially inwards. In another embodiment, it is envisioned that the method described above may be accomplished without the use of the compression ring 226, for example, the conduits 212 may alternatively be displaced or deflected by hand or through the use of a separate tool when positioning the crimping accessory 210 into and out of the transcatheter valve prosthesis 100.

The transcatheter valve prosthesis 100 may then be partially crimped with the leaflets 109 positioned radially inwards via crimping accessory 210. During crimping, the conduits 212 maintain the radial inward position of the leaflets 109 by continuing to apply the suction force thereto. Damage to the leaflets 109 is most likely to occur at the beginning of the crimping process, as there is significant risk of displacement of the leaflets 109 as the stent 102 first begins to be compressed. The crimping accessory 210 is removed after the transcatheter valve prosthesis 100 is partially crimped. As used herein, “partially crimped” includes configurations radially compressed up to 90% of the crimped configuration suitable for delivery within a vasculature, which may also be referred to herein as a fully crimped configuration.

Once the transcatheter valve prosthesis is partially completed, the applied vacuum or suctioned force is discontinued or stopped in order to decouple the conduits 212 from the leaflets 109. After the conduits 212 are no longer coupled to the leaflets 109, the crimping accessory 210 is removed from the transcatheter valve prosthesis 100 and the transcatheter valve prosthesis 100 will then be further crimped to the fully crimped configuration. Removal of the crimping accessory 210 allows the transcatheter valve prosthesis 100 to be radially compressed to a smaller diameter, since the conduits 212 of the crimping accessory 210 are no longer disposed radially within the transcatheter valve prosthesis 100.

In an embodiment, the crimping accessory 210 may be constructed as a portion of the delivery system 201. In greater detail, the crimping accessory 210 may be able to move axially relative to the delivery system 201, so that prior to the crimping process the crimping accessory 210 may be moved into position, and, after the crimping process is complete, the crimping accessory 210 can be moved axially as to not affect the function of the delivery system 201. In other embodiments, the crimping accessory 210 may be placed on the delivery system 201 using a breakaway connection, wherein a portion of the crimping accessory 210 is designed to be weak enough to allow for a user to “break off” the crimping accessory 210 once the crimping process is complete. The breakaway design only allows for the crimping accessory 210 to be used a singular time and may only be used for crimping a single transcatheter valve prosthesis 100.

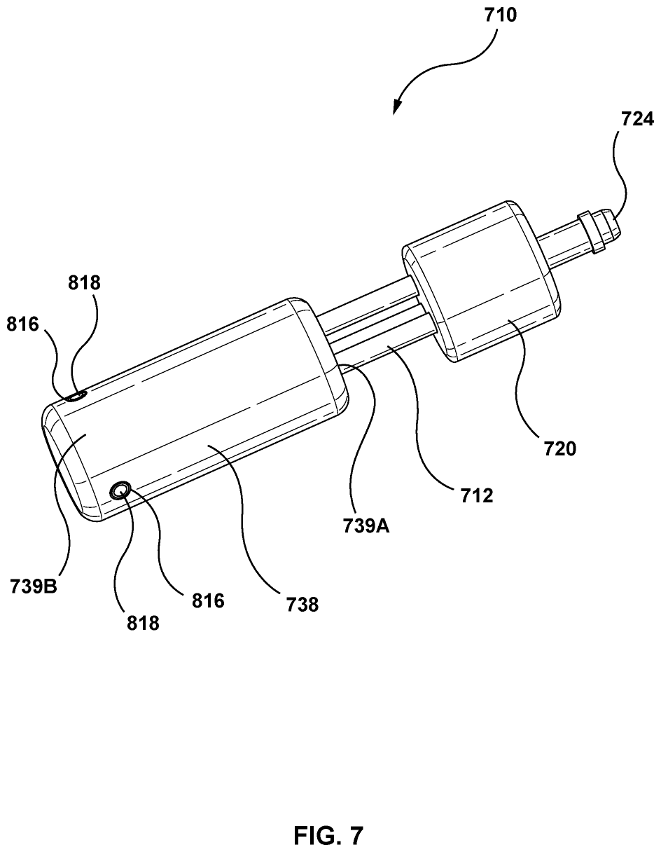

The embodiment described above and the use of the compression ring 226 for deflecting the conduits 212 radially inwards is only exemplary and other structures/components may be used to transition the crimping accessory 210 between the open and closed configurations. For example, a second embodiment of a crimping accessory 710 is shown in FIGS. 7-12. The crimping accessory 710 includes a manifold 720, a plurality of conduits 712 coupled to and extending distally from the manifold 720, and a collar 738 disposed over the conduits 712. The collar 738 of the crimping accessory 710 is configured to guide or direct the conduits 712 between the open and closed configurations, and operates differently than the compression ring 226 as will be explained in more detail below.

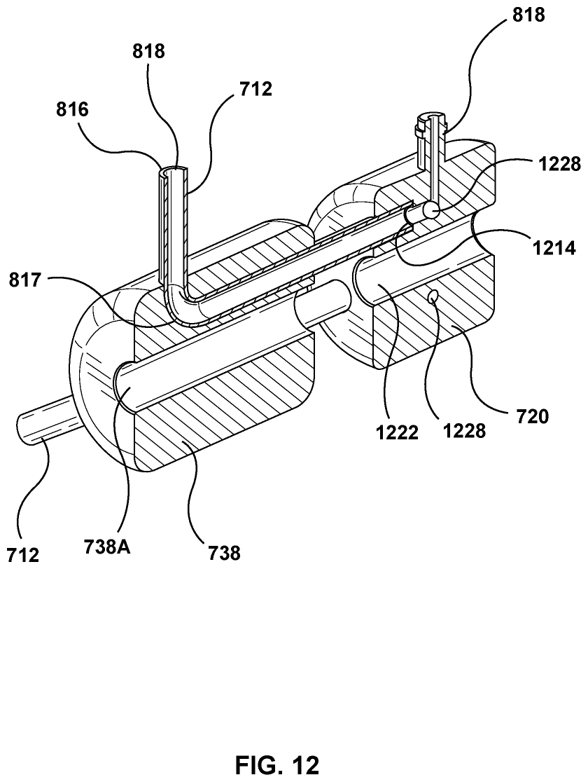

Generally, similar to the manifold 220, the manifold 720 is configured to provide a suction or vacuum force to the conduits 712, which subsequently applies the suction or vacuum force to the leaflets 109. More particularly, the manifold 220 is a cylindrical body that includes a lumen 1222 extending therethrough. The lumen 1222 of the manifold 720 is configured as to allow for the manifold 720 to be slidingly positioned over a delivery system (not shown in FIGS. 7-12). The manifold 720 also includes a coupler 724 which is configured to fluidly connect the crimping accessory 710 to an outside or external vacuum source (not shown in FIGS. 7-12). As best shown on FIG. 12, which shows a sectional view of the crimping accessory 710, the manifold 720 additionally includes a plurality of internal channels 1228 that extend through the manifold 720 as to fluidly couple the coupler 724 to each conduit 712.

Each conduit 712 has a first end 1214 which is disposed within and secured to the manifold 720 and a second end 816 which includes a vacuum port 818 which is configured to contact a leaflet 109 of the transcatheter valve prosthesis 100. Additionally, each conduit 712 includes a lumen which extends from the first end 1214 to the second end 816 allowing for each conduit 712 to apply the suction force to the leaflets 109. Each conduit 712 is configured to contact an inner surface 109A of the leaflets 109 to apply the suction force as to move or displace the leaflets 109 radially inward and away from the side openings 107 of the stent 102. Particularly, the second end 816 of each conduit 712 is configured to contact and hold, using a vacuum or suction force, the inner surface 109A of a leaflet 109 and apply the force as to move or displace the leaflet 109 radially inward and away from the side openings 107 of the stent 102.

Similar to conduits 212, each conduit 712 is formed of a flexible material. Suitable materials for the plurality of conduits 712 include but are not limited to polyimide, Nitinol, or thin-walled stainless steel. The plurality of conduits 712 are circumferentially spaced apart from each other. In an embodiment, the plurality of conduits 712 include at least three conduits. As shown in the embodiment of FIGS. 7-12, the plurality of conduits 712 may include exactly three conduits 712, with each conduit being configured to contact a single leaflet 109 of the valve component 108. However, in another embodiment, the plurality of conduits 712 may include more than three conduits as described above with respect to conduits 212.

A single conduit 712 is shown in FIG. 11, with the conduit 712 being removed from the crimping accessory 710 for sake of illustration only. Each conduit 712 includes a preformed bend or curve 817 along a midportion thereof. As will be explained in more detail below, the collar 738 includes a plurality of collar channels 740 and each collar channel 740 includes an integral bend or curve that matches or corresponds to the preformed bend 817 of the conduit 712.

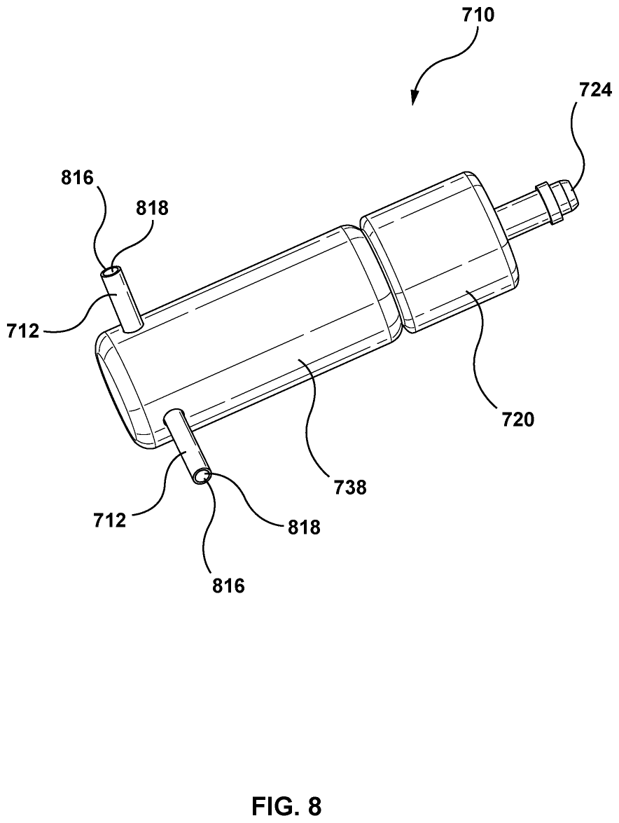

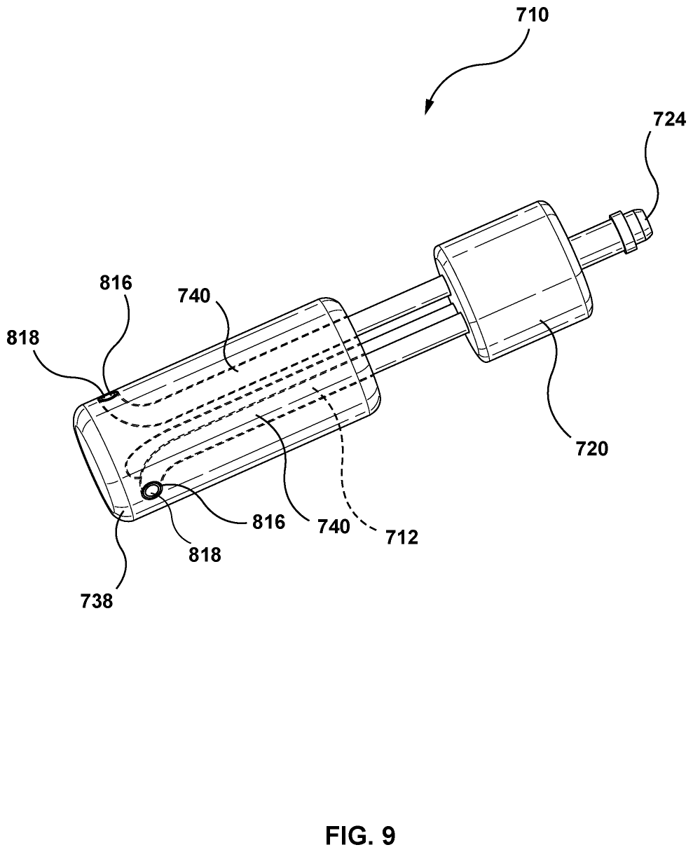

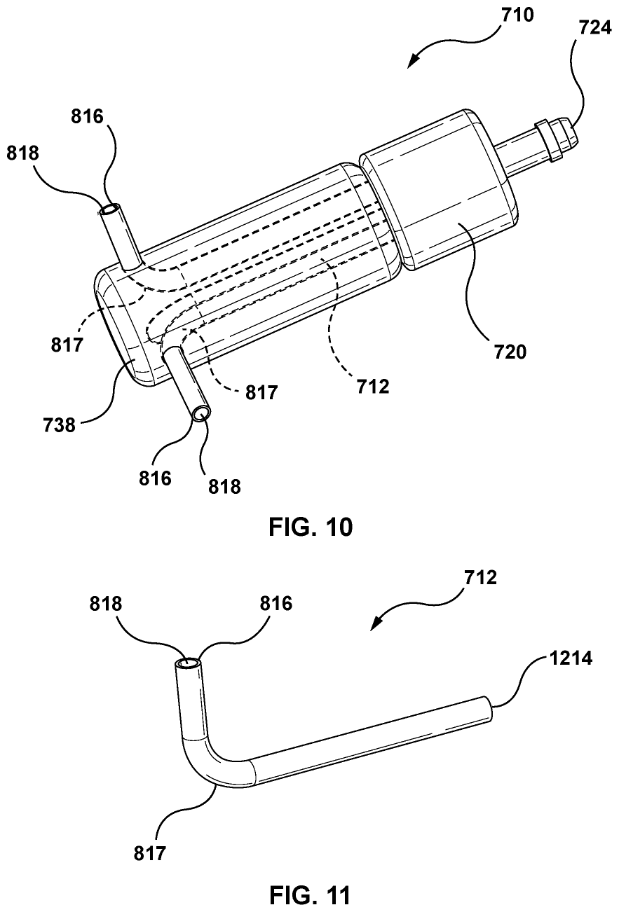

The crimping accessory 710 is configured to have an open configuration, which is shown in FIG. 8 and FIG. 10, and a closed configuration, which is shown in FIG. 7 and FIG. 9. The collar 724 is slidingly disposed over the conduits 712 and is configured to transition the crimping accessory 710 between the open and closed configurations. Stated a different way, axial movement of the collar 738 controls whether the crimping accessory 710 is in the open configuration or the closed configuration. The collar 738 is a tubular component that includes a central lumen 738A which extends through the entirety of the collar 738, the central lumen 738A being sized to accommodate a delivery device therethrough. The diameter of the lumen 1222 of the manifold 720 and the diameter of the central lumen 738A of the collar 738 may be substantially similar and are axially aligned as to allow for the delivery device to extend through both lumens 1222/738A simultaneously.

The collar 738 also includes a plurality of collar channels 740 formed in a sidewall thereof. In the depicted embodiment, each collar channel 740 is configured to house and slidingly receive a single conduit 712. However, this is merely exemplary, and the number of conduits situated within a collar channel 740 may vary. Each collar channel 740 extends from a proximal end 739A of the collar 738 to a curved outer surface 739B of the collar 738. Stated another way, an inlet of each collar channel 740 is disposed at the proximal end 739A of the collar 738 and an outlet of each collar channel 740 is disposed at the curved outer surface 739B of the collar. In an embodiment, the outlet of each collar channel 740 is closer to a distal end of the collar 738 than the proximal end 739A of the collar 738. Each collar channel 740 includes an integral bend or curve 817 that matches or corresponds to the preformed bend 817 of the conduit 712. Proximal to the integral bend or curve, the collar channel 740 extends substantially parallel to the central lumen 738A of the collar 738 and distal to the integral bend or curve, the collar channel 740 extends substantially perpendicular to the central lumen 738A of the collar 738. In other words, each collar channel has a first longitudinal portion that extends within the collar 738 in a first direction and a second longitudinal portion that extends within the collar 738 in a second direction that is perpendicular to the first direction.

While the crimping accessory 710 is in the open configuration, as shown in FIG. 8 and FIG. 10, the conduits 712 extend radially outward from the collar 738 such that the second ends 816 thereof are radially spaced apart from the curved outer surface 739B of the collar 738. Further, the collar 738 is axially spaced adjacent to the manifold 720 and preformed bend 817 of each conduit 712 is disposed within the integral bend or curve 817 of the corresponding collar channel 740. While in the open configuration, the crimping accessory 710 is configured to initially grab or contact the leaflets 109 of the prosthetic valve component 108.

While the crimping accessory 710 is in the closed configuration, as shown in FIG. 7 and FIG. 9, the second end 816 of each conduit 712 is positioned within or adjacent to the outlet of the corresponding collar channel 740 and the collar 738 is axially spaced apart from the manifold 720. While in the closed configuration, the crimping accessory 710 is configured to displace and hold the leaflets 109 radially inward relative to the stent 102. After making contact with the leaflets 109, the crimping accessory 710 may be transitioned from the open configuration to the closed configuration prior to initiation of the crimping process as to move the leaflets 109 away from the stent 102 and, more particularly, the side openings 107 of the stent 102. Alternatively, the crimping accessory 710 may be transitioned from the open configuration to the closed configuration in a gradual manner, during the crimping process. In other words, the crimping accessory 710 may begin in the open configuration at the start of the crimping process and, as the process continues, be transitioned to the closed configuration at a rate that matches the rate of the crimping process.

Referring to the internal views shown in FIG. 9 and FIG. 10, the crimping accessory 710 transitions between the open and closed configurations via relative axial movement between the collar 738 and the conduits 712. In an embodiment, the user moves the assembly of the manifold 720 and the conduits 712 while the collar 738 remains stationary. Sliding the manifold 720 distally forward would push or extend the conduits 712 out of the collar 738, such that the conduits 712 extend radially from the collar 738 in the open configuration. After contact is established and maintained between the leaflets 109 and the conduits 712, via suction, the manifold 720 would be proximally retracted to pull the conduits 712 and the leaflets 109 back towards the collar 738. However, in another embodiment, relative movement between the collar 738 and the conduits 712 may be accomplished via movement of the collar 738 while the assembly of the manifold 720 and the conduits 712 is held stationary. As the collar 738 is proximally retracted towards the manifold 720, the conduits 712 follows the change of direction or curve of the collar channels 740 and the second end 816 of each conduit 712 extends radially out of the collar channels 740. As the collar 738 is distally advanced away from the manifold 720, the conduits 712 are pulled backed into the collar 738. Methods of using the crimping accessory 710 are similar to the methods of using the crimping accessory 210 as described above with reference to FIG. 6.

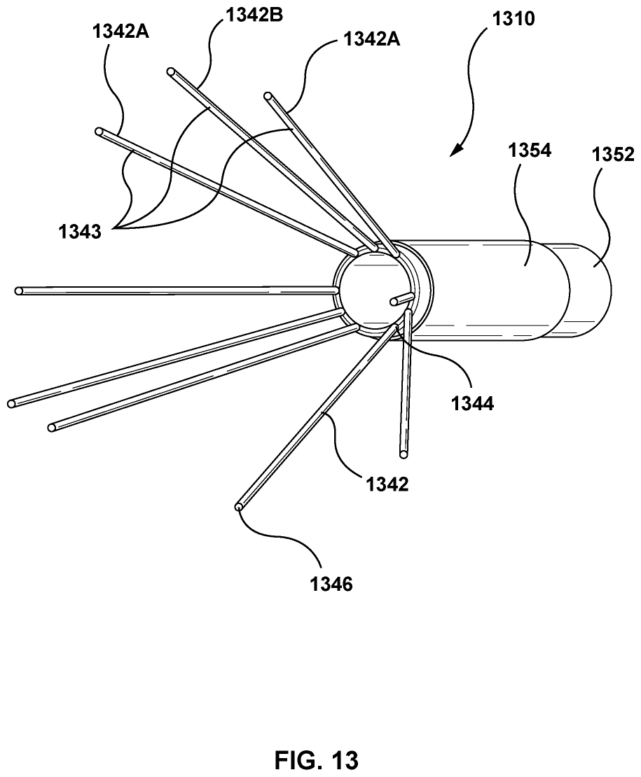

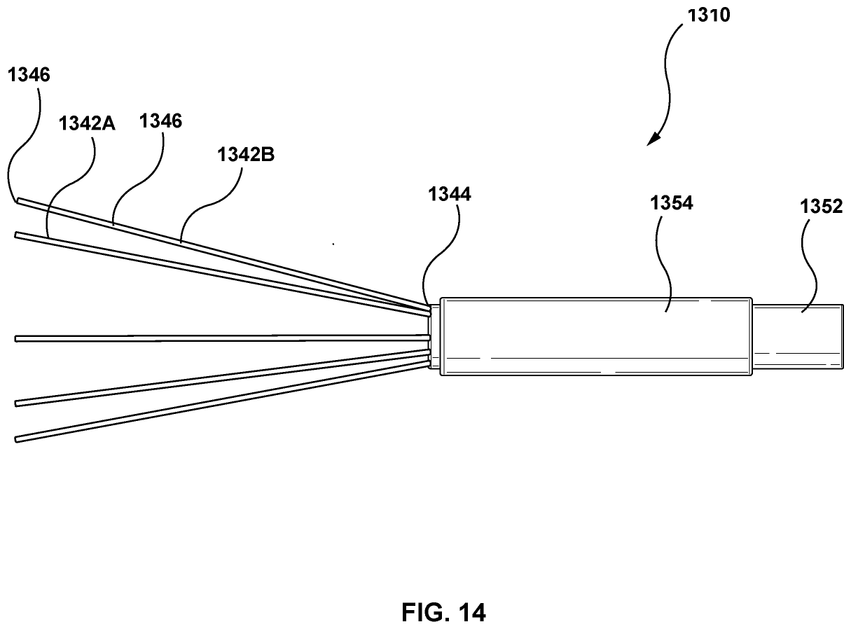

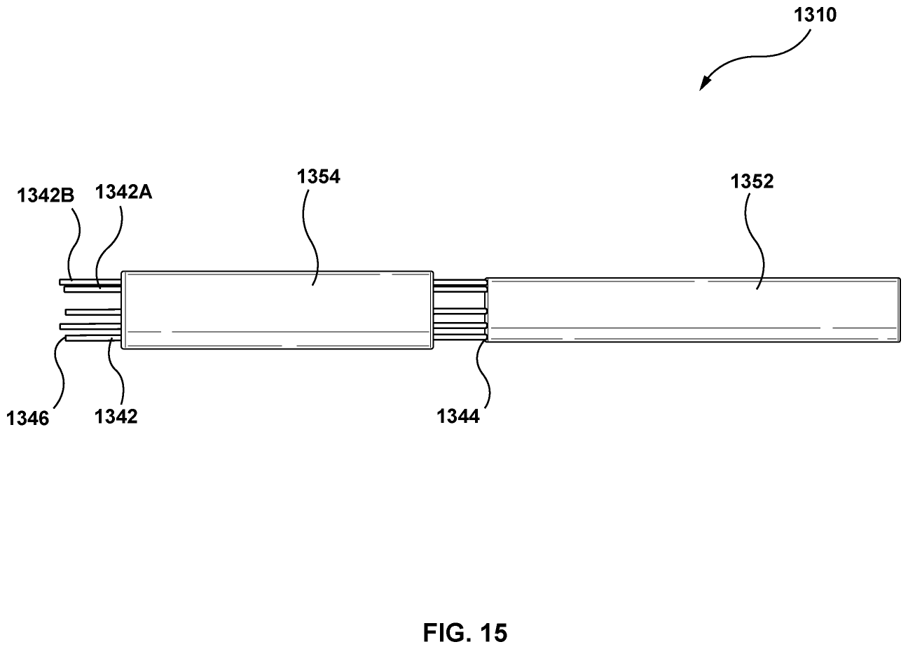

While the previous embodiments utilize suction to maintain contact between the conduits and the leaflets, other embodiments described herein mechanically grasp the leaflets without the use of suction. Another embodiment of a crimping accessory 1310 is shown in FIGS. 13-15. The crimping accessory 1310 includes an open configuration as shown in FIG. 13 and FIG. 14, and a closed configuration configured to grasp the leaflets 109 as shown in FIG. 15. The crimping accessory 1310 includes a plurality of rods 1342, a hub 1352, and a compression sleeve 1354. The hub 1352 is positioned at a proximal end of the crimping accessory 1310 and is configured to be held by a user. Each rod 1342 includes a first end 1344 secured to and distally extending from the hub 1352 and a second end 1346 configured to contact the leaflets 109. Axial movement of the compression sleeve 1354 is configured to transition the crimping accessory 1310 between the open configuration and the closed configuration.

To expand on the plurality of rods 1342, while in the closed configuration each rod 1342 is positioned substantially axially parallel to the hub 1352 and the compression sleeve 1354. While in the open configuration, each rod 1342 is configured to flare or extend radially outward relative to the hub 1352 and the compression sleeve 1354 such that the second end 1346 thereof is positioned radially away from the hub 1352. Each rod is hingedly coupled to the hub 1352 as to allow for the rod 1342 to pivot about the first end 1344. In an embodiment, each rod 1342 is preformed to be biased to the open configuration. In the exemplary embodiments shown in FIGS. 13-15, exactly nine rods 1342 are shown, however, this is merely exemplary and more or less rods may be envisioned with either inner or outer surface orientations.

In order to place a radial force onto the leaflets 109, the crimping accessory 1310 is configured to individually grasp each leaflet 109. More particularly, the plurality of rods 1342 may be considered to include or consist of three groups 1343 of rods 1342, with each group 1343 being configured to grasp an individual leaflet 109. As shown in FIG. 14 and FIG. 15, each group 1343 of rods 1342 includes two inner rods 1342A and a single outer rod 1342B that expands a greater radial distance relative to the hub 1352 than the two inner rods 1342A. The inner rods 1342A and the outer rod 1342B are collectively referred to herein as rods 1342. The different radial positioning of the outer rod 1342B relative to the inner rods 1342 allows a leaflet to be grasped between the rods as the rods transition from the open configuration to the closed configuration. Particularly, each group 1343 of rods 1342 is configured to contact both sides or surfaces of the leaflet 109, with the inner rods 1342A contacting the inner surface 109A of the leaflet 109 and the outer rod 1342B contacting the outer surface 109B of the leaflet 109. As shown in FIG. 15, when in the closed configuration, both the inner rods 1342A and the outer rod 1342B are positioned at the same radial distance relative to the hub 1352. As such, the leaflet 109 is effectively sandwiched between or grasped within the outer rod 1342B and the inner rods 1342 after the rods 1342 have been deflected or displaced to the closed configuration.

As stated above, the compression sleeve 1354 is slidingly disposed over the hub 1352 and is configured to transition the crimping accessory 1310 between the open configuration and the closed configuration when moved axially relative to the hub 1352. The compression sleeve 1354 defines a lumen therethrough that allows for the compression sleeve 1354 to slide over both the hub 1352 and the first ends 1344 of each rod. The axial position of the compression sleeve 1354 relative to the hub 1352 controls whether the crimping accessory 1310 is in the open configuration or the closed configuration. As shown in FIGS. 13 and 14, the open configuration occurs when the compression sleeve 1354 is axially close relative to the hub 1352, while the closed configuration occurs when the compression sleeve 1354 is axially distant relative to the hub 1352 as shown in FIG. 15.

In operation, the crimping accessory 1310 in its open configuration is initially positioned relative to the transcatheter valve prosthesis 100 such that the inner rods 1342A are disposed adjacent to the inner surface 109A of the leaflet 109 and the outer rod 1342B is disposed adjacent to the outer surface 109B of the leaflet 109. The crimping accessory 1310 is then transitioned to its closed configuration such that the rods 1342 are displaced radially inward. More particularly, the compression sleeve 1354 is moved axially away relative to the hub 1352 as to deform the rods 1342 radially inward, and, subsequently, forcing the leaflets 109 radially inward reducing the risk of damage. When the rods 1342 move radially inward, the rods 1342 are able to grasp the leaflets 109, with each leaflet 109 being sandwiched between the inner rods 1342A contacting the inner surface 109A of the leaflet 109 and the outer rod 1342B contacting the outer surface 109B of the leaflet 109. After the leaflets 109 are displaced radially inward, the transcatheter valve prosthesis 100 may then be partially crimped. Once the transcatheter valve prosthesis 100 is partially crimped, the crimping accessory 1310 is proximally retracted in order to decouple the rods 1342 from the leaflets 109. Even after being deflected radially inwards, the rods 1342 can slide or move axially relative to the partially crimped transcatheter valve prosthesis 100. After the rods 1342 are no longer coupled to the leaflets 109, the crimping accessory 1310 is removed from the transcatheter valve prosthesis 100 and the transcatheter valve prosthesis 100 may be fully crimped.

In an embodiment, the rods 1342 may be integrally formed with the hub 1352 as a single component. Alternatively, the rods 1342 may be hingedly attached to the hub 1352.

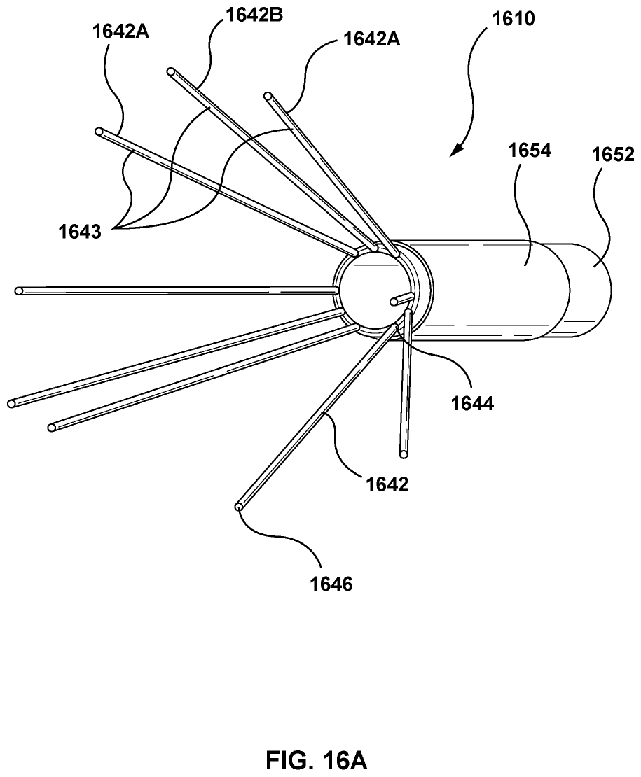

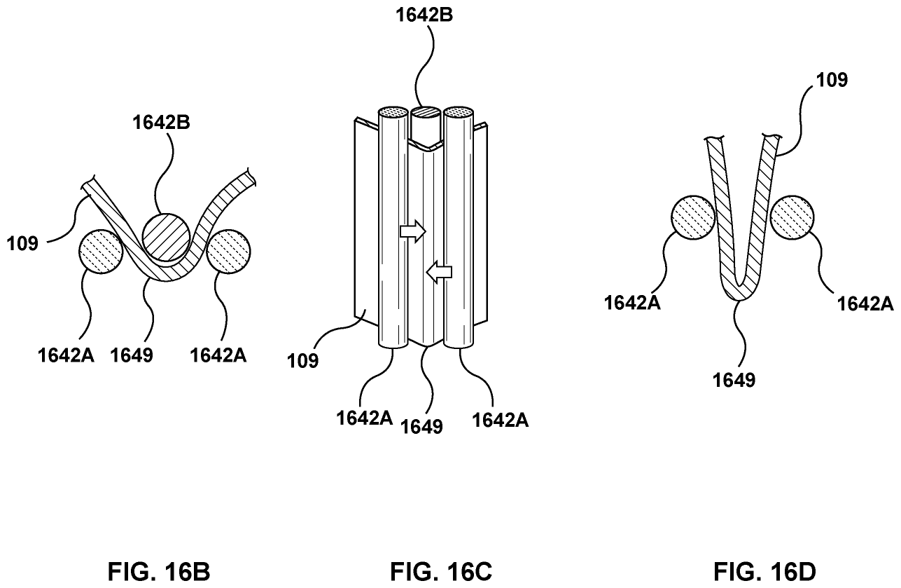

While not required, it is envisioned that the outer rod 1342B of the crimping accessory 1310 may be removable prior to crimping the transcatheter valve prosthesis 100. In such an embodiment, the transcatheter valve prosthesis 100 may be partially crimped without the presence of the outer rod 1342B. The removability of the outer rod 1342B minimizes the overall profile of the crimping accessory and permits the transcatheter valve prosthesis 100 to be partially crimped to a smaller profile with the crimping accessory still disposed within the transcatheter valve prosthesis 100. For example, FIGS. 16A-16D depicts a crimping accessory 1610 which includes removable outer rods 1642B. FIG. 16A depicts a perspective view of the crimping accessory 1610 while FIGS. 16B-16D depict the crimping accessory 1610 grasping a leaflet 109. Similar to the crimping accessory 1310, the crimping accessory 1610 includes a plurality of rods 1642, a hub 1652, and a compression sleeve 1654. The hub 1652 is positioned at a proximal end of the crimping accessory 1610 and is configured to be held by a user. Each rod 1642 includes a first end 1644 secured to and distally extending from the hub 1652 and a second end 1646 configured to contact the leaflets 109. Axial movement of the compression sleeve 1654 is configured to transition the crimping accessory 1610 between the open configuration and the closed configuration.

The crimping accessory 1610 is the same as the crimping accessory 1310, except that the outer rods 1642B are removable from the hub 1652. More particularly, the plurality of rods 1642 may be considered to include or consist of three groups 1643 of rods 1642, with each group 1643 being configured to grasp an individual leaflet 109. Each group 1643 of rods 1642 includes two inner rods 1642A (which are not removable from the hub 1652) and a single removable outer rod 1642B that expands a greater radial distance relative to the hub 1652 than the two inner rods 1642A. The inner rods 1642A and the removable outer rod 1642 are collectively referred to herein as rods 1642. In the exemplary embodiment shown in FIG. 16A, exactly six inner rods 1642A and exactly three removable outer rods 1642B are shown, however this is merely exemplary and more or less rods 1642 may be envisioned.

Each removable outer rod 1642B is removably coupled to the hub 1652 through the use of screws, magnets, or other methods known in the art. For example, in an embodiment, each removeable outer rod 1642B is removably coupled to the hub 1652 via a screw-type mechanism. Each removable outer rod 1642B may include a threaded portion or die extending from the first end 1644 thereof and the hub 1652 may include a hole or opening with inner threads for receiving the corresponding threaded portion. In another embodiment, each removable outer rod 1642B includes a magnet on the first end 1644 thereof and the hub 1652 may include a bore hole with a magnet or magnetic material therein to receive and couple to the magnet of the removable outer rod 1642B. Other examples of mechanisms to removably couple each removable outer rod to the hub includes a press-fit connection in which the removable outer rod may be inserted into a smooth bore hole on the hub of almost equal size as to create a tight fit via friction, or a spring mechanism similar to an alligator clip in which the main spring is disposed on the removable outer rod and the corresponding receiver groove or slot is disposed on the hub.

FIGS. 16B-16D depict how the crimping accessory 1610 uses the two inner rods 1642A and one removable outer rod 1642B in order to fold a leaflet 109. In further detail, and as shown in a top view of FIG. 16B and a side view in FIG. 16C, the two inner rods 1642A are positioned on or adjacent to the inner surface 109A of the leaflet 109 and the removable outer rod 1642B is positioned on or adjacent to the outer surface 109B of the leaflet 109. As the rods 1642 begin to deflect radially inward due to axial movement of the compression sleeve 1654, the leaflet 109 is folded around the removable outer rod 1642B. Once an integral wave or fold 1649 is created on the leaflet 109, the removable outer rod 1642B may be removed while the two inner rods 1642A remain grasping the leaflet 109 around the integral fold 1649 thereof. At this stage of operation, the crimping accessory 1610 is able to grasp each leaflet 109 using only two inner rods 1642A contacting only the inner surface 109A of the leaflet. In another embodiment (not shown), the removable outer rod 1642B may be omitted from the crimping accessory 1610 and the integral fold 1649 may be created on the leaflet using a separate tool as an alternative to the removable outer rod 1642B.

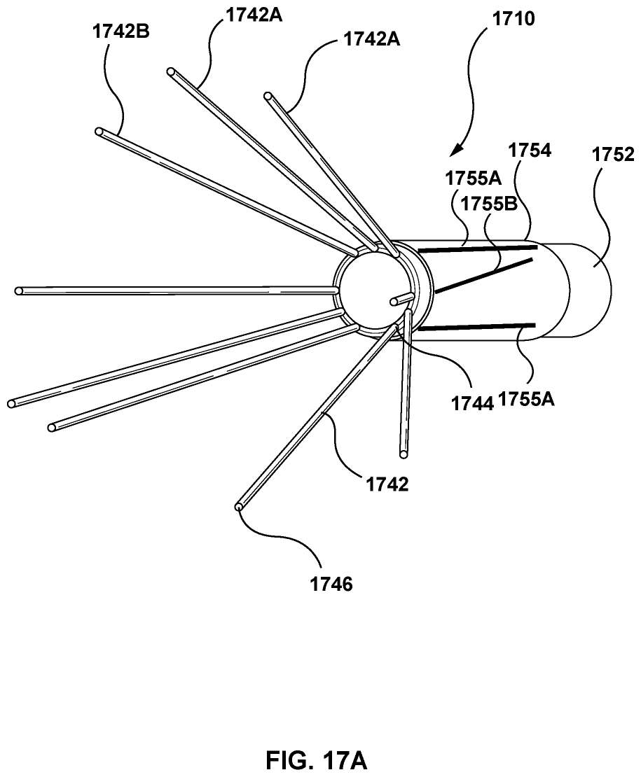

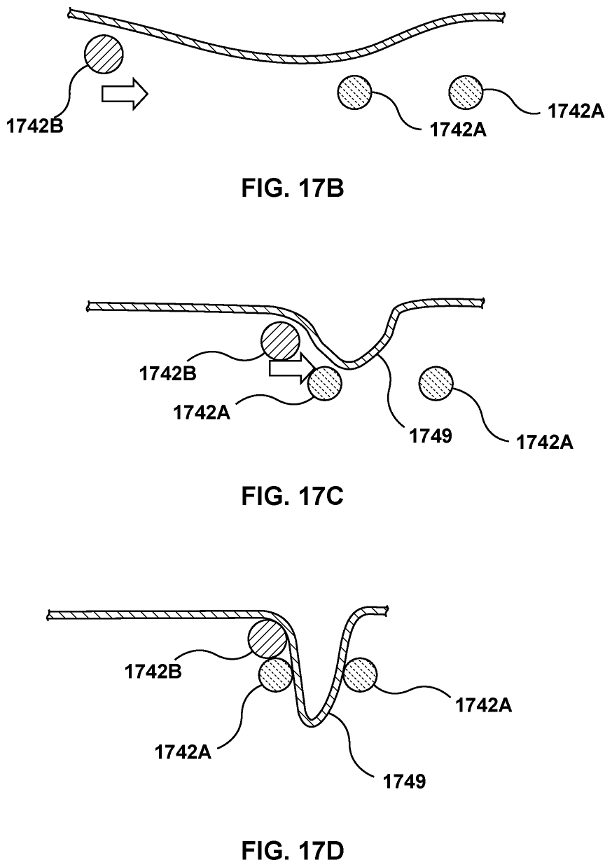

In another embodiment depicted in FIGS. 17A-17D, all rods 1742 of a crimping accessory 1710 are configured to contact an inner surface of a leaflet and one or more rods are configured to create an integral fold 1749 in the leaflet similar to the integral fold 1649 created by the aforementioned crimping accessory 1610. FIG. 17A depicts a perspective view of the crimping accessory 1710 while FIGS. 17B-17D depict the crimping accessory 1710 grasping a leaflet 109. Similar to the crimping accessory 1310, the crimping accessory 1710 includes a plurality of rods 1742, a hub 1752, and a compression sleeve 1754. The hub 1752 is positioned at a proximal end of the crimping accessory 1710 and is configured to be held by a user. Each rod 1742 includes a first end 1744 secured to and distally extending from the hub 1752 and a second end 1746 configured to contact the leaflets 109. Axial movement of the compression sleeve 1754 is configured to transition the crimping accessory 1710 between the open configuration and the closed configuration. The hub 1752 is the same as the hub 1352, and the compression sleeve 1754 is the same as the hub 1354, and thus these components are not described herein in detail.

To expand on the plurality of rods 1742, while in the closed configuration each rod 1742 is positioned substantially axially parallel to the hub 1752 and the compression sleeve 1754. While in the open configuration, each rod 1742 is configured to flare or extend radially outward relative to the hub 1752 and the compression sleeve 1754 such that the second end 1746 thereof is positioned radially away from the hub 1752. Each rod is hingedly coupled to the hub 1752 as to allow for the rod 1752 to pivot about the first end 1744. In an embodiment, each rod 1742 is preformed to be biased to the open configuration. In the exemplary embodiment shown in FIG. 17A, exactly nine rods 1742 are shown, however, this is merely exemplary and more or less rods may be envisioned.

In order to place a radial force onto the leaflets 109, the crimping accessory 1710 is configured to individually grasp each leaflet 109. More particularly, in this embodiment, the plurality of rods 1742 may be considered to include or consist of three groups 1743 of rods 1742, with each group 1743 being configured to grasp an individual leaflet 109. All rods 1742 are configured to contact only the inner surface 109A of the leaflet 109. Each group 1743 of rods 1742 includes two stationary rods 1742A and a single movable rod 1742B that is configured to move relative to the two stationary rods 1742A. The stationary rods 1742A and the movable rod 1742 are collectively referred to herein as rods 1742. In the exemplary embodiment shown in FIG. 17A, exactly six stationary rods 1742A and exactly three movable rods 1742B are shown, however this is merely exemplary and more or less rods may be envisioned.

Each movable rod 1742B is configured to move or travel laterally relative to the stationary rods 1742A as the rods 1742 transitions from the open configuration to the closed configuration. More particularly, the compression sleeve 1754 includes a plurality of tracks 1755A, 1755B formed on an inner surface thereof. As the inner surface of the compression sleeve 1754 is not visible on FIG. 17A, the tracks 1755A, 1755B are shown as thick lines on the compression sleeve 1754 for illustrative purposes only. It will be understood by those of ordinary skill in the art that these thick lines illustrate the placement or trajectory of the tracks but that the tracks are formed on the inner surface of the compression sleeve 1754 for interacting with the rods 1742. Each track is a groove that corresponds to a rod 1742, such that the rod 1742 is slidingly received within the corresponding track. Tracks 1755A are configured to receive stationary rods 1742A, and tracks 1755B are configured to receive moveable rods 1742B. Tracks 1755A corresponding to stationary rods 1742A are substantially straight or linear (i.e., substantially parallel to a longitudinal axis of the compression sleeve 1754), so that the stationary rods 1742A do not move laterally as the hub 1752 (and rods 1742 attached thereto) is retracted or pulled through the compression sleeve 1754. Conversely, tracks 1755B corresponding to the movable rods 1742B are angled relative to the longitudinal axis of the compression sleeve 1754, so that at least the second ends 1746 of the moveable rods 1742 are moved or deflected towards the stationary rods 1742A as the hub 1752 (and rods 1742 attached thereto) is retracted or pulled through the compression sleeve 1754. Stated another way, the movement or positioning of the movable rods 1742B is controlled by the angled tracks in the compression sleeve 1754 to direct or deflect the movable rods 1742B in a lateral direction, towards the stationary rods 1742A. In another embodiment, the straight tracks 1755A for the stationary rods 1742A may be omitted as it is not critical for the stationary rods 1742A to be guided and the compression sleeve 1754 only includes the angled tracks 1755B configured to direct or deflect the movable rods 1742B as desired. Further, in another embodiment, rather than forming the tracks 1755A, 1755B as grooves on the inner surface of the compression sleeve 1754, the tracks 1755A, 1755B may be formed via integral protrusions or rails formed on the inner surface thereof for guiding or deflecting the moveable rods 1742B as desired, i.e., closer to the stationary rods 1742A, as the hub 1752 is retracted or pulled through the compression sleeve 1754.

FIGS. 17B-17D depict how the two stationary rods 1742A and the single movable rod 1742B may be utilized to grasp a single leaflet 109. In order to grasp the leaflet, all rods 1742 are first placed at the leaflet with the crimping accessory 1710 in the open configuration, as depicted in FIG. 17B. As the crimping accessory 1710 is transitioned towards the closed configuration, the movable rods 1742B are directed or deflected across the inner surface 109A of the leaflet 109 towards the two stationary rods 1742A to create an integral wave or fold 1749 on the leaflet 109 as depicted in 17C. As the moveable rod 1742B moves or slides across the surface of the leaflet 109, the friction between the movable rod 1742B and the leaflet 109 causes the leaflet to bunch up between the two stationary rods 1742B. Stated another way, the integral fold or wave 1749 of the leaflet 109 is positioned as to have one of the stationary rods 1742 positioned on either side thereof, as depicted by FIG. 17D. Thus, in this embodiment, the integral fold 1749 is created and the leaflet is thereby grasped by the rods 1742 as the crimping accessory 1710 is transitioned from the open configuration to the closed configuration, deflecting all rods 1742 and the leaflets 109 grasped thereby radially inwards. In another embodiment (not shown), the moveable rods 1742B may be omitted from the crimping accessory 1710 and the integral fold may be created on the leaflet using a separate or independent tool or rod as an alternative to the moveable rod 1742B. Additionally, the stationary rods 1742A and/or the moveable rods 1742B may be configured to alternatively contact the outer surface 109B of the leaflet 109 to create an integral fold 1749 thereon.

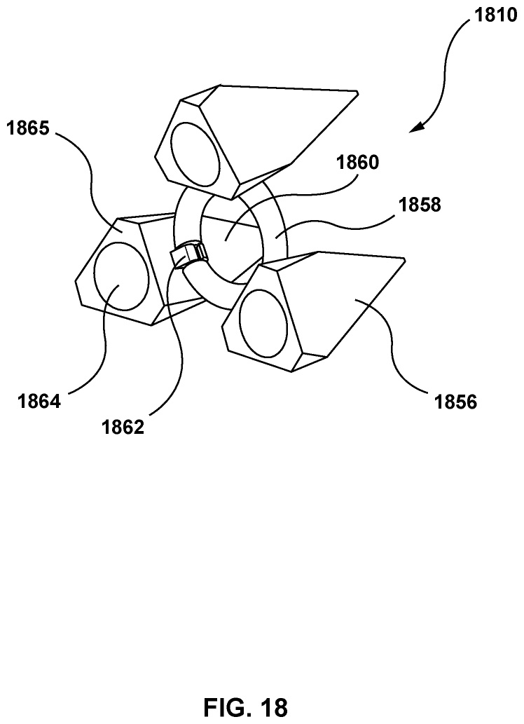

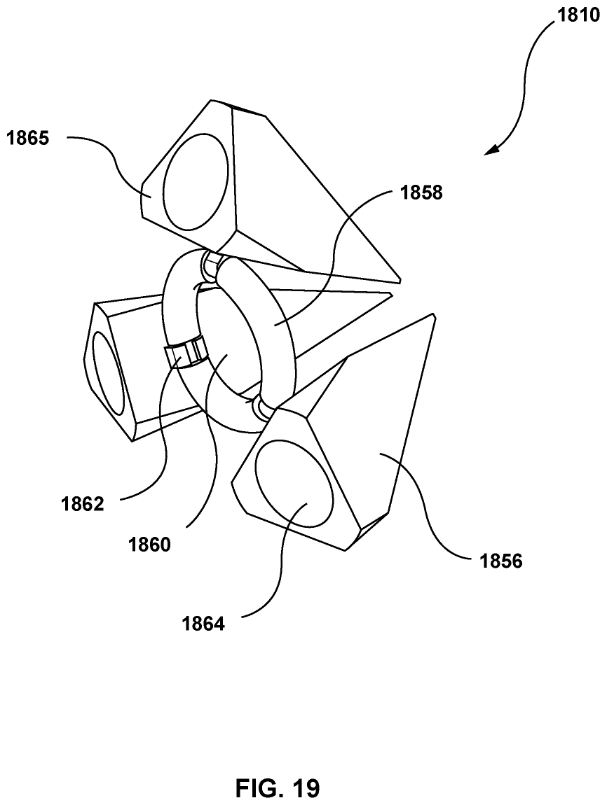

Another embodiment of a crimping accessory 1810 is shown in FIGS. 18-20. The crimping accessory 1810 includes an open configuration, as shown in FIG. 18, and a closed configuration, as shown in FIG. 19. The crimping accessory 1810 includes a body 1858 and a plurality of graspers 1856 coupled to the body 1858. The body 1858 is annular or generally ring shaped, with each of the plurality of graspers 1856 being positioned at circumferentially equal distances around the body 1858.

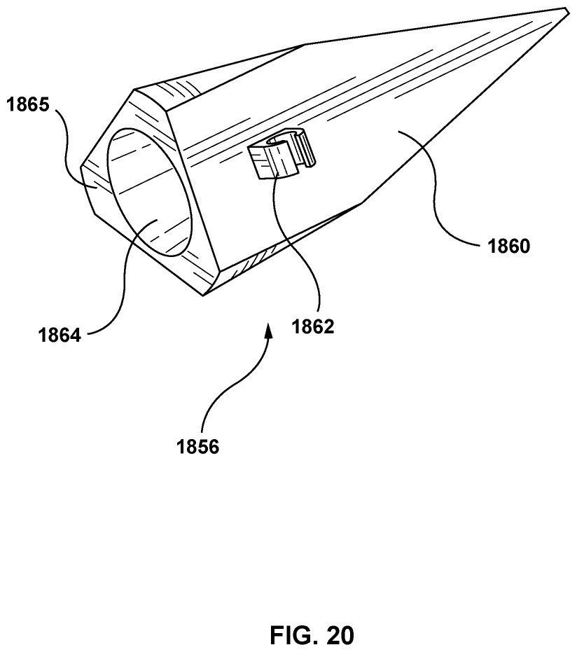

An individual grasper 1856 is shown in FIG. 20. The graspers 1856 are coupled to the body 1858 such that they may each individually pivot relative to the body 1858. Each grasper 1856 is shaped like a three-sided pyramid, with each grasper 1856 including a grasping surface 1860, a connector 1862, and a recess 1864. It is envisioned that each grasper 1856 is configured to interact with a single leaflet 109 of the plurality of leaflets 109. Accordingly, the grasping surface 1860 of each grasper 1856 is configured to contact one of the leaflets 109 as to place a radial force upon the leaflet 109. The connector 1862 of each grasper 1856 pivotally couples the grasper 1856 to the body 1858 while allowing each grasper 1856 to be independently pivoted relative to the body 1858. The recess 1864 of each grasper 1856 is positioned at a base 1865 of the grasper 1856. In some embodiments, the recess 1864 is configured to receive a digit (i.e., a fingertip or thumb) of a user and allow for the user to pivotally control each grasper 1856 using their digits. While exactly three graspers 1856 are shown in FIG. 18 and FIG. 19, this is merely exemplary and it is envisioned that more or less graspers 1856 may be used.

In operation, while the crimping accessory 1810 is in the open configuration, the graspers 1856 are configured to be positioned between the stent 102 and the leaflets 109, i.e., radially inward of the stent 102 and radially outward of the plurality of leaflets 109. Once positioned, the user may pivotally rotate each of the graspers 1856 independently or simultaneously radially inward, as to transition the crimping accessory 1810 to the closed configuration. Once in the closed configuration, each of the graspers 1856 will hold a leaflet 109 radially inward and away from the stent 102. To explain further, each of the graspers 1856 includes the grasping surface 1860 that is configured to contact the outer surface 109B of the leaflet 109, and, as the crimping accessory 1810 is transitioned between the open configuration and the closed configuration, the grasping surface 1860 will come in contact with and radially push in the leaflet 109. As noted above, positioning the leaflets 109 inward reduces the risk of damage to the leaflets 109 during the crimping process of the stent 102. Once the transcatheter valve prosthesis 100 is partially crimped, the crimping accessory 1810 may be removed, and the transcatheter valve prosthesis 100 may be fully crimped.

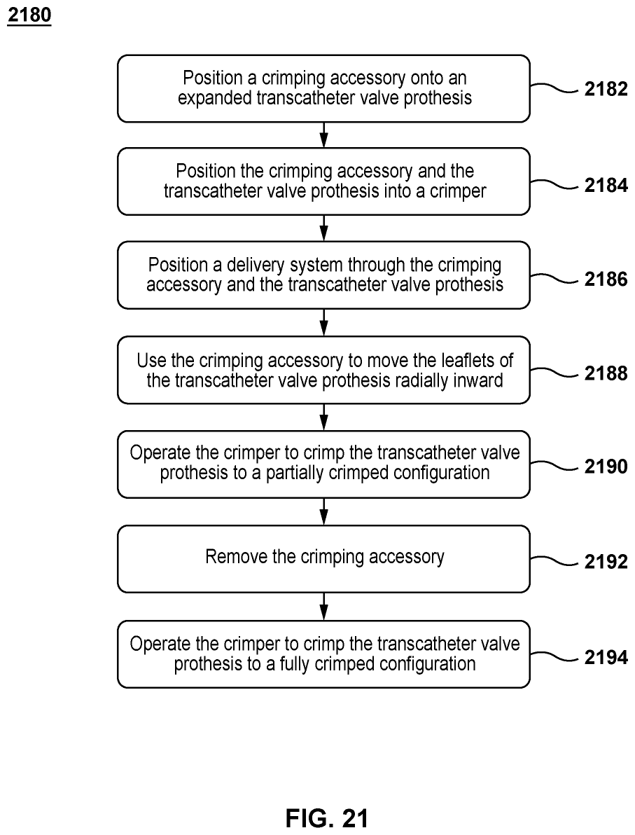

FIG. 21 depicts a method 2180 including the steps 2182-2194 according to an embodiment hereof for using the crimping accessory 210 during crimping of the transcatheter valve prosthesis 100 onto the delivery system 201. Although the method steps of FIG. 21 are described herein with reference to the crimping accessory 210, similar method steps may be performed by the crimping accessory 710, the crimping accessory 1310, the crimping accessory 1610, the crimping accessory 1710, the crimping accessory 1810, or other crimping accessories within the scope of this application.

With reference to step 2182 of FIG. 21, prior to crimping the transcatheter valve prosthesis 100, the crimping accessory 210 is positioned radially inward of the stent 102 of the transcatheter valve prosthesis 100 such that the conduits 212 are disposed radially within the leaflets 109. The plurality of conduits 212 of the crimping accessory 210 are advanced until the second end 216 of the conduits 212 is substantially aligned with the leaflets 109. The placement of the conduits 212 may vary based on a variety of factors, including the size of the leaflets 109, the design of the second end 216 of the conduits, and/or the number of conduits 212 of the crimping accessory 210.

With reference to step 2184 of FIG. 21, the crimping accessory 210 and the transcatheter valve prosthesis 100 is positioned into the crimper chamber 637 of the crimper 636. It will be understood by one of ordinary skill in the art that the crimping accessory 210 and the transcatheter valve prosthesis 100 may be advanced onto the crimper chamber 637 or the crimper 636 may be positioned around the transcatheter valve prosthesis 100 such that the crimper 636 circumferentially surrounds the transcatheter valve prosthesis 100.

With reference to step 2186 of FIG. 21, the delivery system 201 is positioned through the subassembly of the transcatheter valve prosthesis 100 and the crimping accessory 210. The delivery system 201 is disposed as desired longitudinally relative to the transcatheter valve prosthesis 100.

With reference to step 2188 of FIG. 21, the crimping accessory 210 is operated to move the leaflets 109 of the transcatheter valve prosthesis 100 radially inwards. More particularly, a vacuum or suction force is applied to couple the second end 216 of each conduit 212 to an inner surface 109A of a leaflet 109. The conduits 212 and leaflets 109 coupled thereto are then deflected radially inwards via actuation of the compression ring 226.

With reference to step 2190 of FIG. 21, the crimper 636 is operated to radially compress the transcatheter valve prosthesis 100 to a partially crimped configuration onto a portion of the delivery system 854. During initial operation of the crimper 636, the crimping accessory 210 remains positioned or disposed radially within the leaflets 109 of the transcatheter valve prosthesis 100, with the second end 216 of each conduit 212 contacting and holding the leaflets 109 of the transcatheter valve prosthesis 100 radially inwards.

With reference to step 2192 of FIG. 21, after the crimper 636 is operated to partially crimp the crimping accessory 210 with the leaflets 109 being displaced radially inwards as described above, the applied vacuum or suctioned force is discontinued or stopped in order to decouple the conduits 212 from the leaflets 109. After the conduits 212 are no longer coupled to the leaflets 109, the crimping accessory 210 is withdrawn and removed from within the transcatheter valve prosthesis 100. At this stage of the method, the crimper 636 is still positioned over the radially compressed transcatheter valve prosthesis 100.

With reference to 2194 of FIG. 21, after removal of the crimping accessory 210, the crimper is operated to further crimp or radially compress the transcatheter valve prosthesis 100 to its fully crimped configuration, which is suitable for delivery within a vasculature. After the transcatheter valve prosthesis 100 crimped onto a balloon of the delivery system 201, the delivery system 201 is removed from the crimper 636. It will be understood by one of ordinary skill in the art that the delivery system 201 may be removed from the crimper 636 or the crimper 636 may be removed from the delivery system 201.

The following detailed description is merely exemplary in nature and is not intended to limit the invention or the application and use of the invention. Although the description of the invention is in the context of the treatment and navigation of a heart valve, the invention may be used where it is deemed useful in other anatomical sites that are not in the heart. For example, the present invention may be applied to other heart valves or venous valves as well. Furthermore, there is no intention to be bound by any expressed or implied theory presented in the preceding technical field, background, brief summary, or the following detailed description.

While various embodiments according to the present invention have been described above, it should be understood that they have been presented by way of illustration and example only, and not limitation. It will be apparent to person skilled in the relevant art that various changes in form and detail can be made therein without departing from the spirit and scope of the invention. Thus, the breadth and scope of the present invention should not be limited by any one of each embodiment discussed herein, and of each reference cited herein, can be used in combination with the features of any other embodiment. All patents and publication discussed herein are incorporated by reference herein in their entirety.

What is claimed is:

1. An assembly comprising;
a transcatheter valve prosthesis including a stent and a prosthetic valve component including at least one leaflet positioned within and secured to the stent, the transcatheter valve prosthesis having a crimped configuration for delivery within a vasculature and an expanded configuration; and
a crimping accessory configured for use when radially compressing the transcatheter valve prosthesis into the crimped configuration, the crimping accessory including:
at least one conduit, each conduit including:
a first end;
a second end having at least one vacuum port configured to contact an inner surface of the at least one leaflet; and
a lumen extending from the first end to the second end; and
a manifold coupled to the at least one conduit, the manifold being configured to be fluidly coupled to a suction source such that the lumen of the at least one conduit and the at least one vacuum port is also fluidly coupled to the suction source,
wherein the at least one conduit is configured to move radially, and
wherein the crimping accessory is configured to apply a suction force to the inner surface of the at least one leaflet, the suction force applying an inward radial force to move the at least one leaflet radially inwards while the transcatheter valve prosthesis is being radially compressed.

2. The assembly of claim 1, wherein the at least one conduit includes a plurality of conduits, and the plurality of conduits are circumferentially spaced apart from each other at substantially equal intervals.

3. The assembly of claim 1 wherein the crimping accessory further includes a compression ring which is slidably disposed over an outer surface of the manifold and is configured to move axially relative to the manifold, and wherein movement of the compression ring relative to the manifold controls radial movement of the at least one conduit.

4. The assembly of claim 1, wherein the second end of the at least one conduit is curved.

5. The assembly of claim 1, wherein the second end of the at least one conduit has a larger diameter than a diameter of a body of the at least one conduit.

6. The assembly of claim 1, wherein the at least one conduit is curved.

7. An assembly comprising:
a transcatheter valve prosthesis including a stent and a prosthetic valve component including at least one leaflet positioned within the stent and secured to the stent, the transcatheter valve prosthesis having a crimped configuration wherein the transcatheter valve prosthesis is crimped to the catheter delivery system and an expanded configuration for deployment within a native heart valve; and
a crimping accessory configured for use when radially compressing the transcatheter valve into the crimped configuration, the crimping accessory including:
a plurality of rods configured to contact and hold the at least one leaflet and to place a radially inward force as to move the least one leaflet radially inwards; and
a hub operably coupled to an end of each of the plurality of rods, wherein each rod of the plurality of rods is configured to move radially relative to the hub.

8. The assembly of claim 7, wherein the plurality of rods includes exactly nine rods.

9. The assembly of claim 7, wherein the hub of the crimping accessory is configured to be coupled to the delivery system while compressing the transcatheter valve prosthesis into the crimped state.

10. The assembly of claim 7, wherein the leaflet folding assembly further includes a compression ring which is slidably disposed over an outer surface of the hub and is configured to move axially relative to the hub, and wherein movement of the compression ring relative to the hub controls radial movement of the plurality of rods.

11. The assembly of claim 10, wherein exactly three rods are configured to hold a single leaflet, the exactly three rods including a first rod, a second rod and a third rod, the second rod being disposed between the first and third rods.

12. The assembly of claim 11, wherein the first and third rods are configured to contact an inner surface of the single leaflet and the second rod is configured to contact an outer surface of the single leaflet.

13. The assembly of claim 12, wherein the second rod is removably coupled to the hub.

14. The assembly of claim 11, wherein the first and second rods are configured to contact an inner surface of the single leaflet and the third rod is configured to contact an outer surface of the single leaflet.

15. The assembly of claim 14, wherein the compression sleeve includes an angled track on an inner surface thereof for receiving the third rod, the angled track being configured to guide and deflect the third rod laterally towards the first and second rods during relative axial movement between the compression sleeve and the hub.

16. An assembly comprising:
a transcatheter valve prosthesis including a stent and a prosthetic valve component including at least one leaflet positioned within the stent and secured to the stent, the transcatheter valve prosthesis having a crimped configuration for delivery within a vasculature and an expanded configuration; and
a crimping accessory configured for use when radially compressing the transcatheter valve prosthesis into the crimped configuration, the crimping accessory including:
a body;
a plurality of graspers positioned at the body, wherein each grasper is configured to contact exactly one leaflet;
where each of the plurality of graspers is positioned at an outer surface of the at least one leaflet, the crimping accessory configured to contact and hold the at least one leaflet radially inward while the transcatheter valve prosthesis is compressed into the crimped configuration.

17. The assembly of claim 16, wherein the plurality of graspers are circumferentially spaced apart from each other at substantially equal intervals.

18. The assembly of claim 16, wherein each grasper of the plurality of graspers further includes a recess, the recess being configured to accept a finger of a user.

19. The assembly of claim 16, wherein each grasper of the plurality of graspers is operatively coupled to the body as to freely rotate relative to the body.

20. The assembly of claim 19, wherein each grasper of the plurality of graspers may rotate independently from one another.
